(19) United States
(12) Patent Application Publication
(10) Pub. No.: US 2013/0195757 A1
(43) Pub. Date: Aug. 1, 2013

(54) SPARC BINDING APTAMERS AND USES THEREOF

(71) Applicants: Kouros Motamed, Irvine, CA (US); Yanshan Dai, Changsha (CN); Vuong Trieu, Agoura Hills, CA (US); Neil Desai, Los Angeles, CA (US)

(72) Inventors: Kouros Motamed, Irvine, CA (US); Yanshan Dai, Changsha (CN); Vuong Trieu, Agoura Hills, CA (US); Neil Desai, Los Angeles, CA (US)

(73) Assignee: ABRAXIS BIOSCIENCE, LLC, Los Angeles, CA (US)

(21) Appl. No.: 13/695,367

(22) PCT Filed: May 2, 2011
(86) PCT No.: PCT/US2011/034797

§ 371 Date: Apr. 8, 2013

Related U.S. Application Data
(60) Provisional application No. 61/330,051, filed on Apr. 30, 2010.

(51) Int. Cl. A61K 47/48

(52) U.S. Cl. CPC A61K 47/48092
USPC 424/1.73; 536/23.1; 536/18.1; 530/391.7; 424/450; 514/44 R; 514/15.2; 514/34; 435/6.12; 424/178.1

(57) ABSTRACT

The invention relates to nucleic acid aptamers having the ability to bind human SPARC protein (osteonectin) with specificity. The invention also relates to compositions including such aptamers, as well as methods of identifying such aptamers. The invention further relates to methods for using such aptamers in treating and diagnosing proliferative diseases such as, e.g., cancer.

### BACKGROUND OF THE INVENTION

Secreted Protein, Acidic, Rich in Cysteines (SPARC), also known as osteonectin, is a 281 amino acid glycoprotein. SPARC has affinity for a wide variety of ligands including cations (e.g., Ca²⁺, Cu²⁺, Fe²⁺), growth factors (e.g., platelet derived growth factor (PDGF), and vascular endothelial growth factor (VEGF)), extracellular matrix (ECM) proteins (e.g., collagen I-V and collagen IX, vitronectin, and thrombospondin-1), endothelial cells, platelets, albumin, and hydroxyapaptite. SPARC expression is developmentally regulated, and is predominantly expressed in tissues undergoing remodeling during normal development or in response to injury (see, e.g., Lane et al., FASEB J., 8, 163-173 (1994)). High levels of SPARC protein are expressed in developing bones and teeth.

SPARC is a matricellular protein upregulated in several aggressive cancers, but is absent from the vast majority of normal tissues (Porter et al., J. Histochem. Cytochem., 43, 791 (1995) and see below). Indeed, SPARC expression is induced among a variety of tumors (e.g., bladder, liver, ovary, kidney, gut, and breast). In bladder cancer, for example, SPARC expression has been associated with advanced carcinoma. Invasive bladder tumors of stage T2 or greater have been shown to express higher levels of SPARC than bladder tumors of stage T1 (or less superficial tumors), and have poorer prognosis (see, e.g., Yamanaka et al., J. Urology, 166, 2495-2499 (2001)). In meningiomas, SPARC expression has been associated with invasive tumors only (see, e.g., Rempel et al., Clincal Cancer Res., 5, 237-241 (1999)). SPARC expression also has been detected in 74.5% of in situ invasive breast carcinoma lesions (see, e.g., Bellahcene, et al., Am. J. Pathol., 146, 95-100 (1995)), and 54.2% of infiltrating ductal carcinoma of the breast (see, e.g., Kim et al., J. Korean Med. Sci., 13, 652-657 (1998)). SPARC expression also has been associated with frequent microcalcification in breast cancer (see, e.g., Bellahcene et al., supra), suggesting that SPARC expression may be responsible for the affinity of breast metastases for the bone. SPARC is also known to bind albumin (see, e.g., Schnitzer, J. Biol. Chem., 269, 6072 (1994)).

Accordingly, there is a need for compositions and methods that take advantage of SPARC's role in disease and, in particular, SPARC's role in some cancers. Aptamers, or chemical antibodies, are single-stranded nucleic acids that specifically bind target proteins with high affinity, exhibit much higher stability than monoclonal antibodies, lack immunogenicity, and could also elicit biological responses. Aptamers differ from other nucleic acid therapies in that they do not directly affect protein expression, but rather modulate the function of target proteins in a way similar to antibodies or small molecule inhibitors (see, e.g. Thiel et al. Oligonucleotides. 2009 September; 19(3): 209-22). Aptamers offer advantages in addition to binding specificity such as amenability to chemical modifications and ease of production (Id.) As such, aptamers that can target SPARC with high specificity and affinity are desirable alternatives to small molecule and antibody-based therapies.

### BRIEF SUMMARY OF THE INVENTION

The invention provides a SPARC binding aptamer comprising a nucleic acid of SEQ ID NOs: 1-11, or a modification or homolog thereof.

In one aspect, the invention provides a composition comprising a SPARC binding aptamer wherein the SPARC binding aptamer comprises a nucleic acid of SEQ ID NOs: 1-11, or a modification or homolog thereof.

In another aspect, the invention provides a method for diagnosing or treating a disease in an animal comprising administering a diagnostically or therapeutically effective amount of a composition comprising a SPARC binding aptamer, wherein the SPARC binding aptamer comprises a nucleic acid of SEQ ID NOs: 1-11, or a modification or homolog thereof.

In another aspect, the invention provides a method of diagnosing a disease or condition in an animal comprising administering to the animal a diagnostically effective amount of a SPARC binding aptamer comprising SEQ ID NOs:1-11; detecting the amount of SPARC binding aptamer present in a particular site or tissue of the animal; and diagnosing that the disease or condition is present if the amount of SPARC binding aptamer present indicates that significantly greater than normal levels of SPARC are present in the particular site or tissue.

In yet another aspect, the invention provides a kit for the detection or treatment of a disease comprising a pharmaceutical formulation and instructions for use of the formulation in the treatment of tumors, wherein the pharmaceutical formulation comprises a SPARC binding aptamer wherein the SPARC binding aptamer comprises a nucleic acid of SEQ ID NOs: 1-11, or 14 or modifications or homologs thereof.

In another aspect, the invention provides a method for identifying a nucleic acid aptamer with high affinity for SPARC, the method comprising (a) preparing a mixture of nucleic acids comprising random candidate nucleic acids; (b) contacting the mixture of nucleic acids with a tagged SPARC peptide; (c) partitioning the nucleic acids with greater affinity for SPARC from the mixture of nucleic acids; (d) amplifying the nucleic acids of (c) to yield a mixture of candidate nucleic acids having increased affinity for SPARC relative to the mixture of (a); (e) repeating steps (b) through (d) with the mixture of candidate nucleic acids amplified in (d); (f) repeating steps (a) through (e) until a mixture of candidate nucleic acids having high affinity for SPARC is obtained; and (g) identifying nucleic acid aptamers with high affinity for SPARC from the mixture of (f). Such method can also be executed using surface plasmon resonance technology.

In another aspect, the invention provides SPARC binding consensus sequences. The invention provides composition comprising a SPARC binding aptamer wherein the SPARC binding aptamer comprises a nucleic acid of the SPARC binding consensus sequence (SEQ ID NO: 14), or a modification or homolog thereof, wherein up to two of the specified bases of the SPARC binding consensus sequence are changed, and wherein binding affinity of the SPARC binding aptamer to SPARC, as measured by K_{d}, is between 10⁻⁶M and 10⁻⁹M.

In yet another aspect, the invention provides a method for diagnosing or treating a disease in a mammal comprising administering a diagnostically or therapeutically effective amount of a composition comprising a consensus SPARC binding aptamer wherein (a) the consensus SPARC binding aptamer comprises a sequence of SEQ ID NO: 14, wherein up to two of the specified bases of the SPARC binding consensus sequence may be changed, and (b) wherein binding affinity of the SPARC binding aptamer to SPARC, as measured by K_{d} is between 10⁻⁶M and 10⁻⁹M.

In all methods and compositions of the present invention, the SPARC binding aptamer can be conjugated to a therapeutic or diagnostic active agent. Suitable animals for administration of the compositions provided by the invention and application of the methods of the invention include, without limitation, human patients.

### BRIEF DESCRIPTION OF THE SEVERAL VIEWS OF THE DRAWING(S)

FIG. 1 is an alignment of 38 sequences of SPARC aptamers after the 6^{th} round of amplification, selection and purification via GST sepharose affinity chromatography.

FIG. 2 is a phylogenetic tree of selected aptamers after the 6^{th} round of amplification and selection graphically illustrating the similarity between sequences.

FIG. 3 depicts the validation of the BIACORE® surface plasmon resonance aptamer selection method via the binding, and subsequent recovery, of a VEGF aptamer to immobilized VEGF protein.

FIG. 4 depicts a schematic overview of the process of selecting SPARC aptamers via surface plasmon resonance

FIG. 5 is an alignment of aptamers selected after seven rounds of amplification and selection via surface plasmon resonance.

FIG. 6 is a phylogenetic tree of aptamers selected via plasmon resonance method, graphically illustrating the similarities among the sequences.

FIG. 7 is a sensorgram depicting the results of a surface plasmon resonance analysis of the binding of Apt178 to a Biol-SPARC immobilized sensorchip.

FIG. 8 is a sensorgram depicting the results of a surface plasmon resonance analysis of the binding of Apt181 to a Biol-SPARC immobilized sensorchip.

FIG. 9 is a sensorgram depicting the results of a surface plasmon resonance analysis of the binding of Apt184 to a Biol-SPARC immobilized sensorchip.

FIG. 10 is a sensorgram depicting the results of a surface plasmon resonance analysis of the binding of Apt189 to a Biol-SPARC immobilized sensorchip.

FIG. 11 is a sensorgram depicting the results of a surface plasmon resonance analysis of the binding of Apt191 to a Biol-SPARC immobilized sensorchip.

FIG. 12 is a sensorgram depicting the results of a surface plasmon resonance analysis of the binding of AptBia207 to a Biol-SPARC immobilized sensorchip.

FIG. 13 is a sensorgram depicting the results of a surface plasmon resonance analysis of the binding of AptBia208 to a Biol-SPARC immobilized sensorchip.

FIG. 14 is a sensorgram depicting the results of a surface plasmon resonance analysis of the binding of AptBia211 to a Biol-SPARC immobilized sensorchip.

FIG. 15 is a sensorgram depicting the results of a surface plasmon resonance analysis of the binding of AptBia216 to a Biol-SPARC immobilized sensorchip.

FIG. 16 is a sensorgram depicting the results of a surface plasmon resonance analysis of the binding of AptBia231 to a Biol-SPARC immobilized sensorchip.

FIG. 17 is a sensorgram depicting the results of a surface plasmon resonance analysis of the binding of Apt191 to a VEGF immobilized sensorchip.

FIG. 18 schematically depicts the projected secondary structure of Apt100, Apt187, and Apt180.

FIG. 19 is a sensorgram depicting the results of a surface plasmon resonance analysis of the binding of Apt100 to a Biol-SPARC immobilized sensorchip.

FIG. 20 is a sensorgram depicting the results of a surface plasmon resonance analysis of the binding of Apt187 to a Biol-SPARC immobilized sensorchip.

FIG. 21 is a sensorgram depicting the results of a surface plasmon resonance analysis of the binding of Apt180 to a Biol-SPARC immobilized sensorchip.

FIG. 22 is a sensorgram depicting the results of a surface plasmon resonance analysis of the binding and dissociation of an Apt180 aptamer modified to delete the 3′ primer to a Biol-SPARC immobilized sensorchip.

FIG. 23 is an sensorgram depicting the results of a surface plasmon resonance analysis of the binding of an Apt180 aptamer modified to delete the 3′ primer and an Apt180 aptamer modified to delete the 5′ primer to a Biol-SPARC immobilized sensorchip

FIG. 24 is a sensorgram depicting the results of a surface plasmon resonance analysis of the binding and dissociation of an Apt180 aptamer having both the 5′ and 3′ primers deleted to a Biol-SPARC immobilized sensorchip.

FIG. 25 is a sensorgram depicting the results of a surface plasmon resonance analysis of the binding and dissociation of an Apt187 aptamer with both the 5′ and 3′ primers deleted to a Biol-SPARC immobilized sensorchip.

FIG. 26 is a sensorgram depicting the results of a surface plasmon resonance analysis of the binding of Biol-SPARC to an Apt180 immobilized sensorchip.

FIG. 27 is a sensorgram depicting the results of a surface plasmon resonance analysis of the binding of Biol-SPARC to an Apt187 immobilized sensorchip.

FIG. 28 is a sensorgram depicting the results of a surface plasmon resonance analysis of the binding of recombinant human SPARC (purified from E. coli) to an Apt180 immobilized sensorchip.

FIG. 29 is a sensorgram depicting the results of a surface plasmon resonance analysis of the binding and dissociation of mouse SPARC to an Apt180 immobilized sensorchip.

FIG. 30 is a sensorgram depicting the results of a surface plasmon resonance analysis indicating that Apt180 does not bind to bovine serum albumin.

FIG. 31 is a sensorgram depicting the results of a surface plasmon resonance analysis of the consecutive binding of Biol-SPARC and anti-SPARC antibody to Apt180 immobilized sensor chip.

FIG. 31 depicts a sequence alignment and a consensus sequence for SPARC binding aptamers obtained thereby (note: in this application referring to a nucleotide “N” or “X” indicates that that nucleotide that can be G, A, T, U or C).

### DETAILED DESCRIPTION OF THE INVENTION

The present invention relates to SPARC binding aptamers, and compositions, methods, and kits comprising such apatamers. The invention further relates to a consensus sequence of SPARC binding aptamers.

### DEFINITIONS

As used herein, the term “aptamer” means functional single-stranded nucleic acids, or oligonucleotides, that bind ligands with high specificity and affinity.

As used herein, the term “SPARC-binding aptamer” means an aptamer which binds SPARC protein with affinities of at least 1 μM (as measured by K_{d}) and binds VEGF165 with at least 10-fold less affinity.

As used herein the term “nucleic acid” or “oligonucleotide” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Other such modifications are well known to those of skill in the art. Thus, the term nucleic acid also encompasses nucleic acids with substitutions or modifications, such as in the bases and/or sugars.

The term “base” encompasses any of the known base analogs of DNA and RNA. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

In addition, as used herein, the term “nucleic acid” includes peptide nucleic acids. Locked nucleic acids (LNA) are a class of nucleic acid analogues in which the ribose ring is “locked” by a methylene bridge connecting the 2′-O atom and the 4′-C atom. LNA nucleosides contain the common nucleobases (T, C, G, A, U and mC) and are able to form base pairs according to standard Watson-Crick base pairing rules. However, by “locking” the molecule with the methylene bridge the LNA is constrained in the ideal conformation for Watson-Crick binding. When incorporated into a DNA oligonucleotide, LNA therefore makes the pairing with a complementary nucleotide strand more rapid and increases the stability of the resulting duplex.

“Analogs” are nucleic acid sequences or amino acid sequences that have a structure similar to, but not identical to, the native compound.

“Derivatives” are compositions formed from the native compounds either directly, by modification, or by partial substitution.

Derivatives or analogs of the nucleic acids or proteins of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present invention, nucleotides are homologous if the nucleic acid sequences are sufficiently similar to allow recombination and/or hybridization under low stringency conditions.

As used herein, the terms “LNA/DNA mixmer” or “mixmer” are used to refer to a nucleic acid that contains at least one LNA unit and at least one RNA or DNA unit (e.g., a naturally-occurring RNA or DNA unit).

A “gapmer” is based on a central stretch of 4-12 base DNA (gap) typically flanked by 1 to 6 residues of 2′-O modified nucleotides (such as, for example, beta-D-oxy-LNA flanks) which are able to act via an RNaseH mediated mechanism to reduce the target sequence's level.

A “headmer” is defined by a contiguous stretch of beta-D-oxy-LNA or LNA derivatives at the 5′-end followed by a contiguous stretch of DNA or modified monomers recognizable and cleavable by the RNaseH towards the 3′-end, and a “tailmer” is defined by a contiguous stretch of DNA or modified monomers recognizable and cleavable by the RNaseH at the 5′-end followed by a contiguous stretch of beta-D-oxy-LNA or LNA derivatives towards the 3′-end. Suitably, in one such “gapmer” embodiment, said subsequence comprises a stretch of 4 nucleotide analogues, such as LNA nucleotide analogues, as defined herein, followed by a stretch of 8 nucleotides, which is followed by a stretch of 4 nucleotide analogues, such as LNA nucleotide analogues as defined herein, optionally with a single nucleotide at the 3′ end.

In one further “gapmer” embodiment, said subsequence comprises a stretch of 3 nucleotide analogues, such as LNA nucleotide analogues, as defined herein, followed by a stretch of 9 nucleotides, which is followed by a stretch of 3 nucleotide analogues, such as LNA nucleotide analogues as defined herein, optionally with a single nucleotide at the 3′ end. Such a design has surprisingly been found to be very effective.

In one further “gapmer” embodiment, said subsequence comprises a stretch of 4 nucleotide analogues, such as LNA nucleotide analogues, as defined herein, followed by a stretch of 8 nucleotides, which is followed by a stretch of 3 nucleotide analogues, such as LNA nucleotide analogues as defined herein, optionally with a single nucleotide at the 3′ end.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

As used herein “a disease targeting antibody” refers to an antibody that increases the accumulation of an agent at a disease site, in particular, at a tumor site by at least 25%, more preferably at least 50%, even more preferably at least 75%, even more preferably at least 100%, even more preferably at least 3 fold, even more preferably at least 5 fold, even more preferably at least 10 fold, even more preferably at least 20 fold, and most preferably at least 100 fold, as determined by any suitable conventional imaging technique or biopsy and chemical analysis.

As used herein, the term “tumor” refers to any neoplastic growth, proliferation or cell mass whether benign or malignant (cancerous), whether a primary site lesion or metastases.

As used herein, the term “cancer” refers to a proliferative disorder caused or characterized by a proliferation of cells which have lost susceptibility to normal growth control. Cancers of the same tissue type usually originate in the same tissue, and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived). Over 200 different types of cancers are known, and every organ and tissue of the body can be affected. Specific examples of cancers that do not limit the definition of cancer can include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. Alternatively, a cancer can be multicentric or of unknown primary site (CUPS).

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative disease) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent” according to the present invention, also includes a radiation therapy agent or a “chemotherapuetic agent.”

As used herein, the term “diagnostic agent” refers to any chemical used in the imaging of diseased tissue, such as, e.g., a tumor.

As used herein, the term “chemotherapuetic agent” refers to an agent with activity against cancer, neoplastic, and/or proliferative diseases, or that has ability to kill cancerous cells directly.

As used herein, “pharmaceutical formulations” include formulations for human and veterinary use with no significant adverse toxicological effect. “Pharmaceutically acceptable formulation” as used herein refers to a composition or formulation that allows for the effective distribution of the nucleic acid molecules of the instant invention in the physical location most suitable for their desired activity.

As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

As used herein “therapeutically effective amount” refers to an amount of a composition that relieves (to some extent, as judged by a skilled medical practitioner) one or more symptoms of the disease or condition in a mammal. Additionally, “therapeutically effective amount” refers to an amount of a composition that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But, it is within the skill of an ordinarily skilled clinician upon the appreciation of the disclosure set forth herein.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent”, according to the present invention, also includes a radiation therapy agent or a “chemotherapeutic agent.”

As used herein, the term “diagnostic agent” refers to any chemical used in the imaging of diseased tissue, such as, e.g., a tumor.

As used herein, the term “chemotherapeutic agent” refers to an agent with activity against cancer, neoplastic, and/or proliferative diseases.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

As used herein the term “alternative therapeutic regimen” or “alternative therapy” (not a first line chemotherapeutic regimen as described above) may include for example, receptor tyrosine kinase inhibitors (for example Iressa™ (gefitinib), Tarceva™ (erlotinib), Erbitux™ (cetuximab), imatinib mesilate (Gleevec™), proteosome inhibitors (for example bortezomib, Velcade™); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, Rapamune™); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rouse sarcoma virus transforming oncogene (SRC) inhibitors, histonedeacetylase inhibitors, small hypoxia-inducible factor inhibitors, hedgehog inhibitors, and TGF-β signalling inhibitors. Furthermore, an immunotherapeutic agent would also be considered an alternative therapeutic regimen. For example, serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Methods of use of chemotherapeutic agents and other agents used in alternative therapeutic regimens in combination therapies, including dosing and administration regimens, will also be known to a one skilled in the art.

Aptamers

The invention provides a SPARC binding aptamer. In some preferred embodiments, the SPARC binding aptamer comprises a nucleic acid of SEQ ID NOs: 1-11, or a modification or homolog thereof. In other embodiments, the aptamer comprises a SPARC binding consensus sequence (SEQ ID NO: 14), or a modification or homolog thereof.

In some preferred embodiments, the aptamer comprises a nucleic acid of SEQ ID NO: 1. In other preferred embodiments, the aptamer comprises a nucleic acid of SEQ ID NO: 2.

The SPARC-binding aptamer can comprise one or more of DNA, RNA, LNA or PNA. In some preferred embodiments, the aptamer is a DNA or RNA molecule. In certain preferred embodiments, the SPARC binding aptamer comprises a DNA molecule having a sequence of any of SEQ ID NOs: 1-11. When the aptamer is an RNA molecule, the thiamine residues present in the aptamer nucleotide sequence are replaced with uracil residues.

In another aspect, the aptamer comprises a nucleic acid of the SPARC binding consensus sequence (SEQ ID NO: 14), or a modification or homolog thereof. Modifications to the SPARC binding consensus sequence are contemplated, particularly wherein up to two residues of the consensus sequence are changed. As above aptamers prepared from the SPARC binding consensus sequence can comprise one or more of DNA, RNA, LNA or PNA. It will be understood that if the aptamer comprises RNA, thymidine bases will be interpreted as uracil bases. However, such change is not necessarily considered a “modification” to the SPARC binding consensus sequence. In any event, binding affinity of the SPARC binding aptamer to SPARC, as measured by K_{d}, is between 10⁻⁶M and 10⁻⁹M.

In some preferred embodiments, aptamers are provided with stability enhancing features or other features having advantageous properties for ease of preparation or use. For example, the SPARC binding aptamers provided by the invention can comprise a gapmer, mixmer, 2′-MOE, phosphorothioate boranophosphate, 2′-O-methyl, 2′-fluoro, terminal inverted-dT bases, PEG, 2′ tBDMS, 2′-TOM, t′-ACE or combinations thereof. In some embodiments, at least one DNA, RNA, LNA or PNA oligonucleotide of the SPARC binding aptamer is modified by the addition of any one of cholesterol, bis-cholesterol, PEG, PEG-ylated carbon nanotube, poly-L-lysine, cyclodextran, polyethylenimine polymer or peptide moieties.

Suitable oligonucleotides for use in accordance with the invention can be composed of naturally occurring nucleobases, sugars and internucleoside (backbone) linkages as well as oligonucleotides having non-naturally-occurring portions which function similarly or with specific improved functions. Fully or partly modified or substituted oligonucleotides are often preferred over native forms because of several desirable properties of such oligonucleotides, for instance, the ability to penetrate a cell membrane, good resistance to extra- and intracellular nucleases, high affinity and specificity for the nucleic acid target.

In some embodiments, the SPARC binding aptamer comprises at least one LNA unit, such as 3, 4, 5, 6, 7, 8, 9, or 10 LNA units, preferably between 4 to 9 LNA units, such as 6-9 LNA units, most preferably 6, 7 or 8 LNA units. The LNA units comprise at least one beta-D-oxy-LNA unit(s) such as 4, 5, 6, 7, 8, 9, or 10 beta-D-oxy-LNA units. In some embodiments, all LNA units can be beta-D-oxy-LNA units, although it is considered that the oligomeric compounds, such as the antisense oligonucleotide, can comprise more than one type of LNA unit. Suitably, the oligomeric compound can comprise both beta-D-oxy-LNA, and one or more of the following LNA units: thio-LNA, amino-LNA, oxy-LNA, ena-LNA and/or alpha-LNA in either the D-beta or L-alpha configurations or combinations thereof.

Embodiments of the invention can comprise nucleotide analogues, such as LNA nucleotide analogues, the subsequence typically can comprise a stretch of 2-6 nucleotide analogues, such as LNA nucleotide analogues, as defined herein, followed by a stretch of 4-12 nucleotides, which is followed by a stretch of 2-6 nucleotide analogues, such as LNA nucleotide analogues, as defined herein. In one embodiment, the oligonucleotides of the instant invention comprise modified bases such that the oligonucleotides retain their ability to bind other nucleic acid sequences, but are unable to associate significantly with proteins such as the RNA degradation machinery. LNAs are not required, but are a preferred embodiment within the scope of the invention. For increased nuclease resistance and/or binding affinity to the target, the oligonucleotide agents featured in the invention can also include 2′-O-methyl, 2′-fluorine, 2′-O-methoxyethyl, 2′-O-aminopropyl, 2′-amino, and/or phosphorothioate linkages and the like. Inclusion of LNAs, ethylene nucleic acids (ENAS), e.g., 2′-4′-ethylene-bridged nucleic acids, and certain nucleobase modifications such as 2-amino-A, 2-thio (e.g., 2-thio-U), G-clamp modifications, can also increase binding affinity to the target.

While deoxyribonucleotide phosphodiester oligonucleotides are suitable for use in accordance with the invention, the invention is not thusly limited. Methylphosphonate oligonucleotides are noncharged oligomers, in which a nonbridging oxygen atom is replaced by a methyl group at each phosphorus in the oligonucleotide chain. The phosphorothioates in the phosphorothioate diastereomer are thought to have improved nuclease stability. In another embodiment involves the hydrogen at the 2′-position of ribose can be replaced by an O-alkyl group, such as a methyl group. These oligonucleotides form high-melting heteroduplexes with targeted mRNA, and induce an antisense effect by a non-RNase H-dependent mechanism.

Suitable oligonucleotides also include embodiments that do not possess the natural phosphate-ribose backbone. Peptide Nucleic Acids (PNAs) are nucleic acid analogues that contain an uncharged, flexible, polyamide backbone comprised of repeating N-(2-aminoethyl)glycine units to which the nucleobases are attached via methylene carbonyl linkers. These oligomers can form very stable duplexes or triplexes with nucleic acids: single or double-strand DNA or RNA. The property of high-affinity nucleic acid binding can be explained by the lack of electrostatic repulsion because of the absence of negative charges on the PNA oligomers. Because PNAs are not substrates for the RNase H or other RNases, the antisense mechanism of PNAs depends on steric hindrance. PNAs can also bind to DNA and inhibit RNA polymerase initiation and elongation, as well as the binding and action of transcription factors, such as nuclear factor KB. PNAs can also bind mRNA and inhibit splicing or translation, initiation, and elongation.

Phosphorodiamidate morpholino oligomers, in which the deoxyribose moiety is replaced by a morpholine ring and the charged phosphodiester intersubunit linkage is replaced by an uncharged phosphorodiamidate linkage, are also suitable for use in accordance with the invention. These oligonucleotides are very stable in biological systems and exhibit efficient antisense activity in cell-free translation systems and in a few cultured animal cell lines.

Another example of a suitable type of oligonucleotide is the N3′→P5′ PN, which result from the replacement of the oxygen at the 3′ position on ribose by an amine group. These oligonucleotides can, relative to their isosequential phosphodiester counterparts, form very stable complexes with RNA and single- or double-stranded DNA. Specificity, as well as efficacy, can be increased by using a chimeric oligonucleotide, in which the RNase H-competent segment, usually a phosphorothioate moiety, is bounded on one or both termini by a higher-affinity region of modified RNA, e.g., a 2′O-alkyloligoribonucleotides. Without being bound by any particular theory, it is thought that substitution not only increases the affinity of the oligonucleotide for its target but reduces the cleavage of nontargeted mRNAs by RNase H.

Aptamers with the above stability-enhancing or other features are contemplated in the present invention. In some embodiments, stability-enhancing features such as those described herein can subsequently be applied to aptamer sequences that have been selected or identified using methods such as those described herein, however, additional testing may be required to confirm that the resulting aptamers retain the desired binding specificity.

Preferably, SPARC binding aptamers of the present invention bind both SPARC found in the blood, e.g. HTI (platelet) SPARC, and SPARC found at a tumor site, e.g. Biol-SPARC. Various methods of determining aptamer binding strength are known to those of ordinary skill in the art.

Compositions

The invention provides a composition comprising a SPARC binding aptamer, as described above. Preferably, the composition is a pharmaceutically acceptable composition comprising a SPARC binding aptamer and a pharmaceutically acceptable carrier.

The compositions of the present invention can further comprise an active agent. In some embodiments, the active agent is a pharmaceutically active therapeutic agent directly able to exert its pharmacological effect. In other embodiments, the active agent is a diagnostic agent. In preferred embodiments, the active agent is a diagnostic or therapeutic active agent conjugated to a tumor-targeting SPARC binding aptamer. It will be understood that some active agents are useful as both diagnostic and therapeutic agents, and therefore such terms are not mutually exclusive.

Compositions of the present invention can be used to enhance delivery of the active agent to a disease site relative to delivery of the active agent alone, or to enhance SPARC clearance resulting in a decrease in blood level of SPARC. In preferred embodiments, the decrease in blood level of SPARC is at least about 10%. In more preferred embodiments, the decrease in blood level of SPARC is at least about 15%, 20%, 25%, 30%, 35%, 40%, 45%, or, most preferably, at least about 50%.

The active agent can be any suitable therapeutic agent or diagnostic agent, such as a chemotherapeutic or anticancer agent. Suitable chemotherapeutic agents or other anticancer agents for use in accordance with the invention include but, are not limited to, tyrosine kinase inhibitors (genistein), biologically active agents (TNF, or tTF), radionuclides (131I, 90Y, 111In, 211At, 32P and other known therapeutic radionuclides), adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus) and derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, and transplatinum.

Other suitable chemotherapeutic agents for use in accordance with invention include, without limitation, antimetabolites (e.g., asparaginase), antimitotics (e.g., vinca alkaloids), DNA damaging agents (e.g., cisplatin), proapoptotics (agents which induce programmed-cell-death or apoptosis) (e.g, epipodophylotoxins), differentiation inducing agents (e.g., retinoids), antibiotics (e.g., bleomycin), and hormones (e.g., tamoxifen, diethylstilbestrol). Further, suitable chemotherapeutic agents for use in accordance with the invention include antiangiogenesis agents (angiogenesis inhibitors) such as, e.g., INF-alpha, fumagillin, angiostatin, endostatin, thalidomide, and the like.

Preferred chemotherapeutic agents include docetaxel, paclitaxel, and combinations thereof “Combinations thereof” refers to both the administration of dosage forms including more than one drug, for example, docetaxel and paclitaxel, as well as the sequential but, temporally distinct, administration of docetaxel and paclitaxel (e.g., the use of docetaxel in one cycle and paclitaxel in the next). Particularly preferred chemotherapeutic agents comprise particles of protein-bound drug, including but not limited to, wherein the protein making up the protein-bound drug particles comprises albumin including wherein more than 50% of the chemotherapeutic agent is in nanoparticle form. Most preferably the chemotherapeutic agent comprises particles of albumin-bound paclitaxel, such as, e.g., Abraxane®. Such albumin-bound paclitaxel formulations can be used in accordance with the invention where the paclitaxel dose administered is from about 30 mg/mL to about 1000 mg/mL with a dosing cycle of about 3 weeks (i.e., administration of the paclitaxel dose once every about three weeks). Further, it is desirable that the paclitaxel dose administered is from about 50 mg/mL to about 800 mg/mL, preferably from about 80 mg/mL to about 700 mg/mL, and most preferably from about 250 mg/mL to about 300 mg/mL with a dosing cycle of about 3 weeks.

Other therapeutic agents also include, without limitation, biologically active polypeptides, antibodies and fragments thereof, lectins, and toxins (such as ricin A), or radionuclides. Suitable antibodies for use as active agents in accordance with the invention include, without limitation, conjugated (coupled) or unconjugated (uncoupled) antibodies, monoclonal or polyclonal antibodies, humanized or unhumanized antibodies, as well as Fab′, Fab, or Fab2 fragments, single chain antibodies and the like. Contemplated antibodies or antibody fragments can be Fc fragments of IgG, IgA, IgD, IgE, or IgM. In various preferred embodiments, the active agent is a single chain antibody, a Fab fragment, diabody, and the like. In more preferred embodiments, the antibody or antibody fragment mediates complement activation, cell mediated cytotoxicity, and/or opsonization.

In addition, the pharmaceutically active agent can be an siRNA. In preferred embodiments, the siRNA molecule inhibits expression of an gene associated with tumors such as, for example, c-S is and other growth factors, EGFR, PDGFR, VEGFR, HER2, other receptor tyrosine kinases, Src-family genes, Syk-ZAP-70 family genes, BTK family genes, other cytoplasmic tyrosine kinases, Raf kinase, cyclin dependent kinases, other cytoplasmic serine/threonine kinases, Ras protein and other regulatory GTPases.

The invention further provides a diagnostic agent conjugated to a SPARC binding aptamer. Suitable diagnostic agents include, e.g., fluorchromes, radioisotopes or radionuclides, MRI contrast agents, X-ray contrast agents, ultrasound contrast agents and PET contrast agents.

The active agent can be coupled to the tumor-targeting SPARC binding aptamer using any method known to one of skill in the art. For example, the SPARC binding aptamer and the active agent can be coupled using a method such as biotin-streptavidin conjugation, chemical conjugation, covalent coupling, antibody coupling, and/or direct expression (e.g., a chimeric protein).

In other embodiments, free amino groups in SPARC binding aptamers can be conjugated with reagents such as carbodiimides or heterobiofunctional agents. In addition, sugar moieties bound to suitable SPARC binding aptamers, can be oxidized to form aldehyde groups useful in a number of coupling procedures known in the art. The conjugates formed in accordance with the invention can be stable, in vivo, or labile, such as enzymatically degradeable tetrapeptide linakages, or acid-labile, cis-aconityl, or hydrazone linkages.

SPARC binding aptamers can also be conjugated to polyethylene glycol (PEG). PEG conjugation can increase the circulating half-life of a protein, reduce the protein's immunogenicity and antigenicity, and improve the bioactivity. Any suitable method of conjugation can be used, including but not limited to, e.g., reacting methoxy-PEG with a SPARC binding aptamer's available amino groups or other reactive sites such as, e.g., histidines or cysteines. In addition, recombinant DNA approaches can be used to add amino acids with PEG-reactive groups to the inventive SPARC binding aptamers. PEG can be processed prior to reacting it with a SPARC binding aptamer, e.g., linker groups can be added to the PEG. Further, releasable and hybrid PEG-ylation strategies can be used in accordance with the invention, such as, e.g., the PEG-ylation of a SPARC binding aptamer such that the PEG molecules added to certain sites in the SPARC binding aptamer are released in vivo. Such PEG conjugation methods are known in the art (See, e.g., Greenwald et al., Adv. Drug Delivery Rev. 55:217-250 (2003)).

Contemplated SPARC binding aptamers and conjugates thereof can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present inventions are generally provided in a formulation with a carrier, such as a pharmaceutically acceptable carrier. Typically, the carrier will be liquid, but also can be solid, or a combination of liquid and solid components. The carrier desirably is a physiologically acceptable (e.g., a pharmaceutically or pharmacologically acceptable) carrier (e.g., excipient or diluent). Physiologically acceptable carriers are well known and are readily available. Suitable pharmaceutical excipients include stabilizers, antioxidants, osmolality adjusting agents, buffers, and pH adjusting agents. Suitable additives include physiologically biocompatible buffers, additions of chelants or calcium chelate complexes, or, optionally, additions of calcium or sodium salts. Pharmaceutical compositions can be packaged for use in liquid form, or can be lyophilized. Preferred physiologically acceptable carrier media are water, buffered water, normal saline, 0.4% saline, 0.3% glycine, hyaluronic acid and the like. The choice of carrier will be determined, at least in part, by the location of the target tissue and/or cells, and the particular method used to administer the composition.

The composition can be formulated for administration by a route including intravenous, intraarterial, intramuscular, intraperitoneal, intrathecal, epidural, topical, percutaneous, subcutaneous, transmucosal (including, for example, pulmonary), intranasal, rectal, vaginal, or oral. The composition also can comprise additional components such as diluents, adjuvants, excipients, preservatives, and pH adjusting agents, and the like.

Formulations suitable for injectable administration include aqueous and nonaqueous, isotonic sterile injection solutions, which can contain anti-oxidants, buffers, bacteriostats, and solutes that render the formulation isotonic with the blood of the intended recipient, and aqueous and nonaqueous sterile suspensions that can include suspending agents, solubilizers, thickening agents, stabilizers, lyoprotectants, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, or tablets.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In all cases, the formulation must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

In preferred embodiments, the active ingredients can be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Specifically, liposomes containing the SPARC binding aptamers can be prepared by such methods as described in Rezler et al., J. Am. Chem. Soc. 129(16): 4961-72 (2007); Samad et al., Curr. Drug Deliv. 4(4): 297-305 (2007); and U.S. Pat. Nos. 4,485,045 and 4,544,545. Liposomes with enhanced circulation time are disclosed in U.S. Pat. No. 5,013,556. Albumin nanoparticles are particularly preferred in the compositions of the present invention.

Particularly useful liposomes can be generated by, for example, the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Polynucleotides of the present invention can be conjugated to the liposomes using methods as described in Werle et al., Int. J. Pharm. 370(1-2): 26-32 (2009).

In other embodiments, a composition can be delivered using a natural virus or virus-like particle, a dendrimer, carbon nanoassembly, a polymer carrier, a paramagnetic particle, a ferromagnetic particle, a polymersome, a filomicelle, a micelle or a lipoprotein.

Administration into the airways can provide either systemic or local administration, for example to the trachea and/or the lungs. Such administration can be made via inhalation or via physical application, using aerosols, solutions, and devices such as a bronchoscope. For inhalation, the compositions herein are conveniently delivered from an insufflator, a nebulizer, a pump, a pressurized pack, or other convenient means of delivering an aerosol, non-aerosol spray of a powder, or noon-aerosol spray of a liquid. Pressurized packs can comprise a suitable propellant such a liquefied gas or a compressed gas. Liquefied gases include, for example, fluorinated chlorinated hydrocarbons, hydrochlorofluorocarbons, hydrochlorocarbons, hydrocarbons, and hydrocarbon ethers. Compressed gases include, for example, nitrogen, nitrous oxide, and carbon dioxide. In particular, the use of dichlorodifluoromethane, trichlorofluoromethane, dichlorotetrafluoroethane, carbon dioxide or other suitable gas is contemplated. In the case of a pressurized aerosol, the dosage unit can be determined by providing a valve to deliver a controlled amount. In administering a dry powder composition, the powder mix can include a suitable powder base such as lactose or starch. The powder composition can be presented in unit dosage form such as, for example, capsules, cartridges, or blister packs from which the powder can be administered with the aid of an inhalator or insufflator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, inhaled aerosols, rectal or vaginal suppositories, mouthwashes, rapidly dissolving tablets, or lozenges. For transdermal administration, the active compounds are formulated into ointments, salves, gels, foams, or creams as generally known in the art.

The pharmaceutical compositions can be delivered using drug delivery systems. Such delivery systems include hyaluronic acid solutions or suspensions of collagen fragments. The drugs can be formulated in microcapsules, designed with appropriate polymeric materials for controlled release, such as polylactic acid, ethylhydroxycellulose, polycaprolactone, polycaprolactone diol, polylysine, polyglycolic, polymaleic acid, poly[N-(2-hydroxypropyl)methylacrylamide] and the like. Particular formulations using drug delivery systems can be in the form of liquid suspensions, ointments, complexes to a bandage, collagen shield or the like.

The composition can further comprise any other suitable components, especially for enhancing the stability of the composition and/or its end-use. Accordingly, there is a wide variety of suitable formulations of the composition of the invention.

Sustained release compositions can also be employed in the present compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the pharmaceutical composition and physiological distress.

Compositions provided by the invention can include, e.g., from about 0.5 mL to about 4 mL aqueous or organic liquids with an active agent coupled to a SPARC binding aptamer, with the concentration of the active agent from about 10 mg/mL to about 100 mg/mL, preferably from about 1 mg/mL to about 10 mg/mL, more preferably from about 0.1 mg/mL to about 1 mg/mL. The active agent can be present at any suitable and therapeutically effective concentration, e.g., Avastin at a concentration of from about 10 mg/mL to about 50 mg/mL.

Methods

In another aspect, the invention provides a method for diagnosing or treating a disease in a mammal comprising administering a diagnostically or therapeutically effective amount of a composition comprising a SPARC binding aptamer. In some embodiments, the invention provides a method for diagnosing a disease in a mammal comprising administering an effective amount of a composition comprising a SPARC binding aptamer. In other embodiments, the invention provides a method for treating a disease in a mammal comprising administering an effective amount of a composition comprising a SPARC binding aptamer. Any SPARC binding aptamer or composition described herein can be used in the methods of the present invention.

According to the methods of the present invention, a therapeutically effective amount of the composition can be administered to the mammal to enhance delivery of the active agent to a disease site relative to delivery of the active agent alone, or to enhance clearance resulting in a decrease in blood level of SPARC. In preferred embodiments, the decrease in blood level of SPARC is at least about 10%. In more preferred embodiments, the decrease in blood level of SPARC is at least about 15%, 20%, 25%, 30%, 35%, 40%, 45%, or, most preferably, at least about 50%.

The invention also provides a method of diagnosing a disease or condition in an animal comprising (a) administering to the animal a diagnostically effective amount of a SPARC binding aptamer comprising SEQ ID NOs:1-11; (b) detecting the amount of SPARC binding aptamer present in a particular site or tissue of the animal; and (c) diagnosing that the disease or condition is present if the amount of SPARC binding aptamer present indicates that significantly greater than normal levels of SPARC are present in the particular site or tissue.

The present methods can be used in any condition characterized by overexpression of SPARC. Exemplary diseases for which the present invention is useful include abnormal conditions of proliferation, tissue remodeling, hyperplasia, exaggerated wound healing in any bodily tissue including soft tissue, connective tissue, bone, solid organs, blood vessel and the like. Examples of diseases treatable or diagnosed using the methods and compositions of the present invention include cancer, diabetic or other retinopathy, inflammation, arthritis, restenosis in blood vessels or artificial blood vessel grafts or intravascular devices and the like.

Other diseases within the scope of the methods of the present invention include, without limitation, cancer, restenosis or other proliferative diseases, fibrosis, osteoporosis or exaggerated wound healing. Specifically, such suitable diseases include, without limitation, wherein: (a) the cancer can be, for example, circinoma in situ, atypical hyperplasia, carcinoma, sarcoma, carcinosarcoma, lung cancer, pancreatic cancer, skin cancer, hematological neoplasms, breast cancer, brain cancer, colon cancer, bladder cancer, cervical cancer, endometrial cancer, esophageal cancer, gastric cancer, head and neck cancer, multiple myeloma, liver cancer, leukemia, lymphoma, oral cancer, osteosarcomas, ovarian cancer, prostate cancer, testicular cancer, and thyroid cancer, (b) the restenosis can be, for example, coronary artery restenosis, cerebral artery restenosis, carotid artery restenosis, renal artery restenosis, femoral artery restenosis, peripheral artery restenosis or combinations thereof, (c) the other proliferative disease can be, for example, hyperlasias, endometriosis, hypertrophic scars and keloids, proliferative diabetic retinopathy, glomerulonephritis, proliferatve, pulmonary hypertension, rheumatoid arthritis, arteriovenous malformations, atherosclerotic plaques, coronary artery disease, delayed wound healing, hemophilic joints, nonunion fractures, Osler-Weber syndrome, psoriasis, pyogenic granuloma, scleroderma, tracoma, menorrhagia, vascular adhesions, and papillomas, and (d) the fibrotic disease can be, for example, hepatic fibrosis, pulmonary fibrosis and retroperitoneal fibrosis.

The animal can be any patient or subject in need of treatment or diagnosis. In preferred embodiments, the animal is a mammal. In particularly preferred embodiments, the animal is a human. In other embodiments, the animal can be a mouse, rat, rabbit, cat, dog, pig, sheep, horse, cow, or a non-human primate.

The invention provides a method for delivering a chemotherapeutic agent to a tumor in a mammal. The methods comprise administering to a human or other animal a therapeutically effective amount of a pharmaceutical composition, wherein the pharmaceutical composition comprises the chemotherapeutic agent coupled to a suitable SPARC binding aptamer and a pharmaceutically acceptable carrier. Descriptions of the chemotherapeutic agents, animals, and components thereof, set forth herein in connection with other embodiments of the invention also are applicable to those same aspects of the aforesaid method of delivering a chemotherapeutic agent to a tumor.

The types of tumor to be detected, whose response to chemotherapy can be predicted or determined, or which can be treated in accordance with the invention are generally those found in humans and other mammals. The tumors can be the result of inoculation as well, such as in laboratory animals. Many types and forms of tumors are encountered in human and other animal conditions, and there is no intention to limit the application of the methods of the present to any particular tumor type or variety. Tumors, as is known, include an abnormal mass of tissue that results from uncontrolled and progressive cell division, and is also typically known as a “neoplasm.” The inventive methods are useful for tumor cells and associated stromal cells, solid tumors and tumors associated with soft tissue, such as, soft tissue sarcoma, for example, in a human.

The tumor or cancer can be located in the oral cavity and pharynx, the digestive system, the respiratory system, bones and joints (e.g., bony metastases), soft tissue, the skin (e.g., melanoma), breast, the genital system, the urinary system, the eye and orbit, the brain and central nervous system (e.g., glioma), or the endocrine system (e.g., thyroid) and is not necessarily limited to the primary tumor or cancer. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, small cell and non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system. The tumor or cancer can be located in the head and/or neck (e.g., laryngeal cancer and parathyroid cancer). The tumor or cancer also can be located in the hematopoietic system or lymphoid system, and include, for example, lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). Preferably, the tumor is located in the bladder, liver, ovary, kidney, gut, brain, or breast.

In other embodiments, the inventive methods comprise administering to a mammal a therapeutically effective amount of a pharmaceutical composition comprising a liposome bound or albumin bound chemotherapeutic agent wherein the liposome or albumin is coupled to a suitable disease targeting SPARC binding aptamer. The chemotherapeutic agent can be coupled to the SPARC binding aptamer using any suitable method. Preferably, the chemotherapeutic agent is chemically coupled to the compound via covalent bonds including, for example, disulfide bonds.

One or more doses of one or more chemotherapeutic agents, such as those described above, can also be administered according to the inventive methods. The type and number of chemotherapeutic agents used in the inventive method will depend on the standard chemotherapeutic regimen for a particular tumor type. In other words, while a particular cancer can be treated routinely with a single chemotherapeutic agent, another can be treated routinely with a combination of chemotherapeutic agents. Methods for coupling or conjugation of suitable therapeutics, chemotherapeutics, radionuclides, etc. to antibodies or fragments thereof are well described in the art.

Methods in accordance with the invention include, e.g., combination therapies wherein the animal is also undergoing one or more cancer therapies selected from the group consisting of surgery, chemotherapy, radiotherapy, thermotherapy, immunotherapy, hormone therapy and laser therapy. The terms “co-administration” and “combination therapy” refer to administering to a subject two or more therapeutically active agents. The agents can be contained in a single pharmaceutical composition and be administered at the same time, or the agents can be contained in separate formulation and administered serially to a subject. So long as the two agents can be detected in the subject at the same time, the two agents are said to be co-administered.

Combination therapies contemplated in the present invention include, but are not limited to antibody administration, vaccine administration, administration of cytotoxic agents, natural amino acid polypeptides, nucleic acids, nucleotide analogues, and biologic response modifiers. Two or more combined compounds may be used together or sequentially. Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interleukin 2 Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

Compositions featured in the methods of the present invention can be administered in a single dose or in multiple doses. Where the administration of the aptamers by infusion, the infusion can be a single sustained dose or can be delivered by multiple infusions. Injection of the agent can be directly into the tissue at or near the site of aberrant target gene expression. Multiple injections of the agent can be made into the tissue at or near the site.

Dosage levels on the order of about 1 ug/kg to 100 mg/kg of body weight per administration are useful in the treatment of a disease. In regard to dosage, an aptamer can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of aptamer per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of aptamer per kg of bodyweight. The unit dose, for example, can be administered by injection (e.g., intravenous or intramuscular, intrathecally, or directly into an organ), inhalation, or a topical application.

One skilled in the art can also readily determine an appropriate dosage regimen for administering the aptamer of the invention to a given subject. For example, the SPARC-binding aptamer composition can be administered to the subject once, as a single injection or deposition at or near the site of SPARC expression. Compositions of the present invention can be administered daily, semi-weekly, weekly, bi-weekly, semi-monthly, monthly, bi-monthly, or at the discretion of the clinician. In some embodiments, the compositions are administered once or twice daily to a subject for a period of from about three to about twenty-eight days, more preferably from about seven to about ten days. In further embodiments, the unit dose is administered less frequently than once a day, e.g., less than every 2, 4, 8 or 30 days. In other embodiments, the unit dose is not administered with a frequency (e.g., not a regular frequency).

Where a dosage regimen comprises multiple administrations, it is understood that the effective amount of SPARC-binding aptamer composition administered to the subject can include the total amount of aptamer administered over the entire dosage regimen. One skilled in the art will appreciate that the exact individual dosages may be adjusted somewhat depending on a variety of factors, including the specific SPARC binding aptamer composition being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. Following successful treatment, it may be desirable to have the patient undergo maintenance therapy to prevent the recurrence of the disease state. The concentration of the aptamer composition is an amount sufficient to be effective in treating or preventing a disorder or to regulate a physiological condition in humans. The concentration or amount of aptamer administered will depend on the parameters determined for the agent and the method of administration.

Certain factors may influence the dosage required to effectively treat a subject, including but not limited to the severity of the disease or disorder, previous treatments, the general health and/or age of the subject, and other diseases present. It will also be appreciated that the effective dosage of the aptamer used for treatment may increase or decrease over the course of a particular treatment. Changes in dosage may result and become apparent from the results of diagnostic assays. For example, the subject can be monitored after administering an aptamer composition. Based on information from the monitoring, an additional amount of the aptamer composition can be administered. Persons of ordinary skill can easily determine optimum dosages, dosing methodologies and repetition rates.

In another aspect, the invention provides a method for identifying a nucleic acid aptamer with high affinity for SPARC, the method comprising (a) preparing a mixture of nucleic acids comprising random candidate nucleic acids; (b) contacting the mixture of nucleic acids with a tagged SPARC peptide; (c) partitioning the nucleic acids with greater affinity for SPARC from the mixture of nucleic acids; (d) amplifying the nucleic acids of (c) to yield a mixture of candidate nucleic acids having increased affinity for SPARC relative to the mixture of (a); (e) repeating steps (b) through (d) with the mixture of candidate nucleic acids amplified in (d); (f) repeating steps (a) through (e) until a mixture of candidate nucleic acids having high affinity for SPARC is obtained; and (g) identifying nucleic acid aptamers with high affinity for SPARC from the mixture of (f). Specific protocols for preparation and identification of aptamers, such as by systematic evolution of ligands by exponential enrichment (SELEX) and/or other methods, are known to one of skill in the art. See, e.g., Mayer, Angew Chem. Int. Ed. Engl. 48(15): 2672-89 (2009), and references cited therein. In some embodiments, GST sepharose affinity chromatography as described herein can be used. In other embodiments, acetylcholine affinity column chromatography can be used. Still other methods will be known to one of ordinary skill in the art. Additional protocols relating to preparation and identification of RNA aptamers in particular are cited in Yan et al., RNA Biol. 6(3):316-20 (2009).

In preferred embodiments, nucleic acid aptamers with high affinity for SPARC can be identified using surface plasmon resonance (SPR) technology. In some embodiments, an SPR instrument such as a BIACORE 3000® (GE/Biacore International AB, Uppsala, Sweden) can be used to execute such methods. However, one of ordinary skill in the art will understand that any suitable SPR instrument can be used.

In one embodiment, the invention provides a method for identifying a nucleic acid aptamer with high affinity for SPARC comprising (a) preparing a sensor chip immobilized with SPARC using an SPR instrument; (b) preparing a mixture of nucleic acids comprising random candidate nucleic acids; (c) injecting the mixture of nucleic acids onto a flow cell wherein the sensor chip is docked in the SPR instrument; (d) partitioning the nucleic acids with greater affinity for SPARC from the mixture of nucleic acids by elution and recovery of bound nucleic acids; (e) amplifying the nucleic acids of (d) to yield a mixture of candidate nucleic acids having increased affinity for SPARC relative to the mixture of (b); (f) repeating steps (c) through (e) with the mixture of candidate nucleic acids amplified in (e); (g) repeating steps (c) through (f) until a mixture of candidate nucleic acids having high affinity for SPARC is obtained, and (h) identifying nucleic acid aptamers with high affinity for SPARC from the mixture of (g).

In the above methods of selecting or identifying SPARC binding aptamers, random candidate nucleotides with stability-enhancing features or other features can be used provided they are compatible with the enzymatic steps of the above inventions. In some embodiments, features compatible with the enzymatic steps of the above that have been described in the art include those described above, and especially 2′-amino, 2′-fluoro, 2′-methoxy, 4′ thiol, 2′-LNA, 5-pentenyl, 5-[3-(pent-4-ynoylamino)prop-1-yn-1-yl], 5-propynylguanidine, 5-(3-aminopropyl) and 1,6-diaminohexyl-N-5-carbamoylmoylmethyl (Mayer, Agnew Chem. Int. Ed. 48: 2672-2689 (2009)). Other stability enhancing features for use in preparing aptamers are also known to those of skill in the art. See, e.g., Keefe et al., Curr. Opn. Chem. Biol. 12: 448-456 (2008). In other embodiments, stability-enhancing features such as the above can be applied to aptamers after they have been selected or identified, however, additional testing may be required to confirm that the resulting aptamers retain the desired binding specificity.

Kits

The invention also provides a kit for the detection or treatment of a disease comprising a pharmaceutical composition of a SPARC binding aptamer and instructions for use of the formulation in the treatment of tumors. In preferred embodiments, the SPARC binding aptamer comprises a nucleic acid of SEQ ID NOs: 1-11, including but not limited to a DNA aptamer having a sequence of any of SEQ ID NOs: 1-11, a nucleic acid comprising a SPARC binding consensus sequence, an RNA aptamer of a SPARC binding consensus sequence wherein thiamine residues are interpreted as uracil, or a modification or homolog of any of the foregoing. Any SPARC binding aptamer or composition described herein can be used in the methods of the present invention. Any SPARC binding aptamer or composition described above can be used in the methods of the present invention.

The following examples further illustrate the invention but, of course, should not be construed as in any way limiting its scope.

### Example 1

This example demonstrates in vitro selection of DNA oligonucleotide sequences that bind to human SPARC protein using GST sepharose affinity chromatography.

A degenerate oligonucleotide library was synthesized at 1 μmol scale (Invitrogen, CA). Each oligonucleotide in this library, designated Apt-Lib-N30, comprises approximately 30 random nucleotides flanked by 18 nt of 5′ and 3′ primer sequences suitable for PCR amplification.

This material was diluted to 0.5 nmol/μL in 10 mM Tris-HCl, at a pH of 7.5, 0.5 mM EDTA.

Forward primer sequence: 5′-CTTAAGCTACCATTGGTT-3′(SEQ ID NO: 15). Reverse primer sequence in both biotinylated and non-biotinylated forms in the 5′-end: 5′-GCTTGGCTAGATCAGATGA-3′(SEQ ID NO: 13).

GST- and GST-SPARC-bound Glutathione-coupled sepharose beads were prepared by incubating protein-containing medium from Sf9 insect cells secreting GST or GST-SPARC. The bead-bound GST and GST-SPARC were then washed 3× with PBS. The protein yield and purity were determined by subjecting the proteins to 10% SDS-PAGE and stained with Coomassie blue, and amounts determined by Bradford assay.

Selection stringency was controlled by adjusting the concentration of target protein, oligonucleotide DNA, NaCl, and by varying incubation time and washing time.

In the first round of selection, 125 nmol of Apt-Lib-N30 library was heated to 95° C. for 5 minutes then cooled to 4° C. for 1 minute. The material was incubated at room temperature for 5 minutes. The material was then incubated with 4.2 nM of GST protein that was bound to Sepharose beads in 10 mL binding buffer, 10 mM Tris-HCl, pH 7.5, 150 mM NaCl in falcon 15 mL conical tube for 1 hour with shaking at room temperature. The tube was centrifuged at 1000×g for 5 minutes. The supernatant was then added to 4.2 nM of GST-SPARC bound beads and incubated at room temperature for 1 hour. The tube was centrifuged at 1000×g for 5 minutes at room temperature. The supernatant was removed. The beads were washed 3 times with 10 mL binding buffer, 2 minutes for each wash at room temperature with shaking. The bound DNA oligonucleotides were eluted with 40 μL of 3 M NaCl, 10 mM EDTA by boiling at 95° C. for 5 minutes.

The eluted bound DNA oligonucleotides were amplified by PCR. 50 μL of PCR reaction contained 1 units Platinum Taq polymerase (Invitrogen, Carlsbad, Calif.), 0.2 μM forward primer and biotinylated reverse primer, 0.2 mM dNTPs, 1.5 mM MgCl2. Amplification conditions were as follows: 3 minutes at 95 C for activation of Taq Polymerase, followed by 20 sec at 95° C., 20 sec at 55° C. and 20 sec at 72° C. for 25 cycles, and 5 minutes at 72° C. Ten μL of PCR were subjected to 2% agarose gel electrophoresis to check the size and quantity of PCR products. About 250 ng of PCR products were generated in each PCR reaction estimated by comparing to DNA ladder. 50 μL of PCR products were then mixed for 30 minutes at room temperature with 1 mg of Streptavidin Dynabeads (Invitrogen Dynal AS, Oslo, Norway) in the binding and washing buffer containing 5 mM Tris-HCl, pH 7.5, 0.5 mM EDTA, 1 M NaCl, then washed 3×1 mL with the washing buffer. Single strand sense strand DNA were separated from the bead bound biotinylated anti-sense strand DNA using a 2 minutes incubation of 50 μl of freshly made 100 mM NaOH. The collected sense strand DNA was neutralized by adding 5 μL of 1 M glacial acetic acid.

The collected single stranded sense DNA was heated to 95° C. for 2 min, then placed on 4° C. for 1 minute. The material was left at room temperature for 5 min, then applied to next round of selection by incubating with 4.2 nM of GST-SPARC in 1 mL binding buffer (10 mM Tris-HCl, pH 7.5 150 mM NaCl). For additional round of selection (rounds 2 to 8), the incubation time was reduced to 10 minutes and wash time was increased to 10 minutes for each wash, total 30 minutes wash time. For rounds 7 and 8, the binding buffer included 10 mM KCl, 2 mM MgCl₂, 2 mM CaCl₂. Counter selection with GST protein bound beads was performed after rounds 3, 6, and 8.

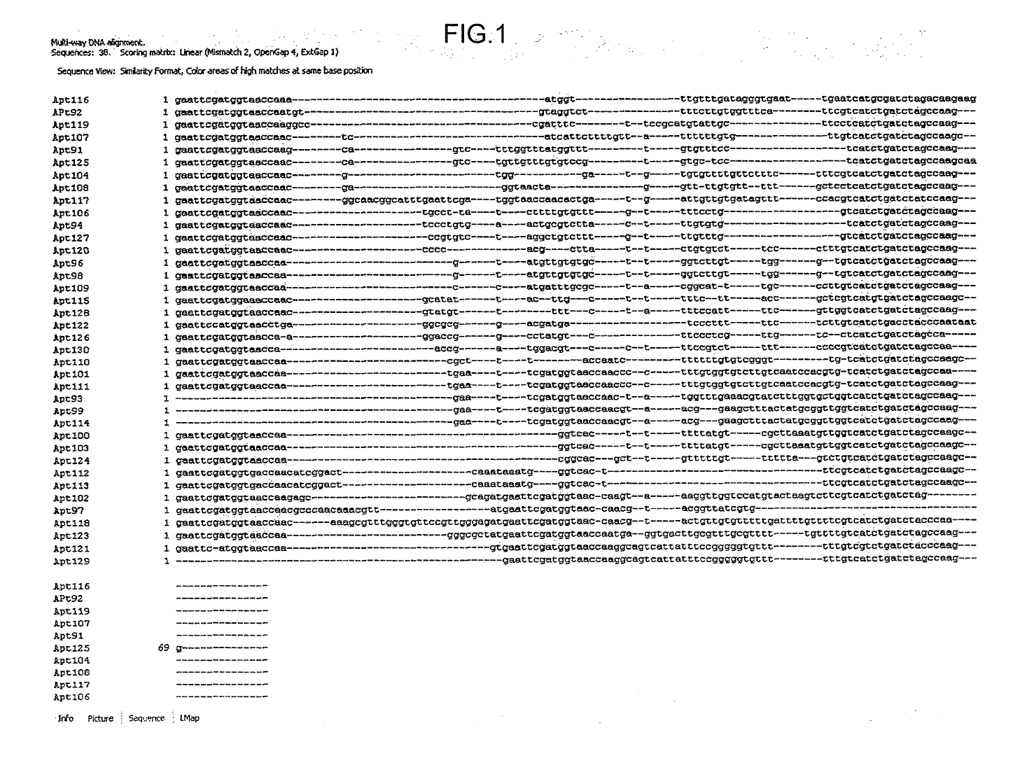

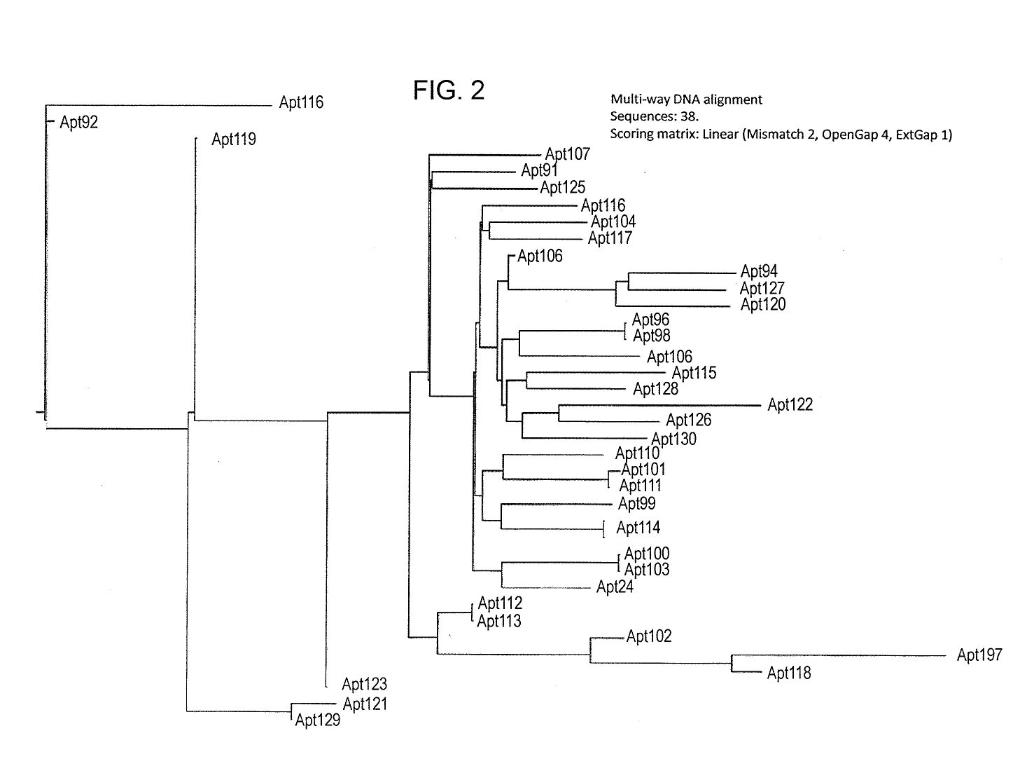

After round 6 and round 8, the eluted oligonucleotide DNA were amplified by PCR with forward and non-biotinylated reverse primer, and PCR products were cloned into pCR4-Topo vector using TOPO TA cloning kit (Invitrogen, Carlsbad, Calif.), and transformed into Top10 DH5α competent cells (Invitrogen, Carlsbad, Calif.). 40 colonies were picked for round 6 and 8, respectively, and sequenced by Laragen (Los Angeles, Calif.). DNA sequences were analyzed and aligned using Clone Manager Professional Suite 8. An alignment of 38 sequences from round 6 is provided in FIG. 1. A phylogenic tree (FIG. 2) graphically illustrates similarity among sequences.

These results show that SPARC binding oligonucleotides can have a variety of sequences which have a range of homology at the primary structural level.

### Example 2

This example demonstrates the selection of SPARC binding DNA aptamers using surface plasmon resonance (SPR) technology.

A BIACORE 3000® (GE/Biacore International AB, Uppsala, Sweden) SPR instrument was used to perform 7 rounds of selection to isolate aptamers that bind to immobilized recombinant human SPARC (Biol). Biol was diluted to 100 μg/mL at 10 mM sodium acetate buffer, pH 4.5 and injected to fc2 or fc4 flow cells, leaving fc1 and fc3 as blank.

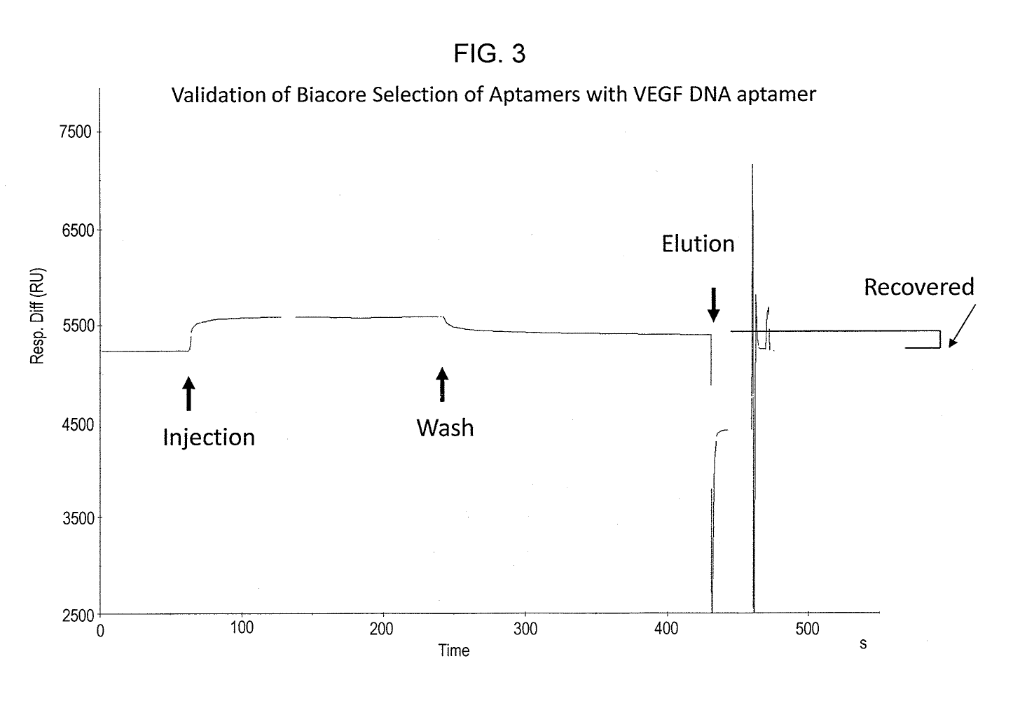

Purified SPARC was immobilized onto the surface of a CM5 biosensor chip (GE/Biacore International AB) by the use of the amine coupling kit and the Surface Preparation tool as present in the BIACORE 3000® control software. The biosensor chip surface was activated by injecting (35 μL at a flow rate of 5 μL/min) a mixture of N-hydroxysuccinimide (NHS) and N-ethyl-N′-(dimethyl-aminopropyl)-carbodiimide (EDC) (1:1; v/v) into one of the four flow channels (Fcs). Then the SPARC, diluted (0.1 mg/mL) in coupling buffer (10 mM sodium acetate, pH 4.5), was injected and bound to the activated carboxymethylated dextran surface via its primary amine groups. After coupling, the remaining active groups were blocked with ethanolamine (1 M). Purified recombinant human VEGF165 (PeproTech, Rocky Hill, N.J.) was diluted into 100 μg/mL in sodium acetate, pH 5.5 and immobilized onto the biosensor chip surface as a control, and VEGF DNA aptamers were injected (FIG. 3). To validate recovery, the resulting bound VEGF aptamers were eluted, and 2 μL were recovered, out of which 0.5 μL was amplified by PCR. Immobilization levels were reached at 6,600 RU (response units).

Operations on the SPR instrument were controlled using Analyte Recovery tool in the BIACORE 3000® software version 4.01 from the Integrated μ-Fluidic Cartridge. HEPES-buffered saline containing 10 mM HEPES, 150 mM NaCl, and 0.005% surfactant P-20 (HBS) was used as running and washing buffer, and regeneration buffer (3 M NaCl, 10 mM EDTA) was used to elute bound aptamers.

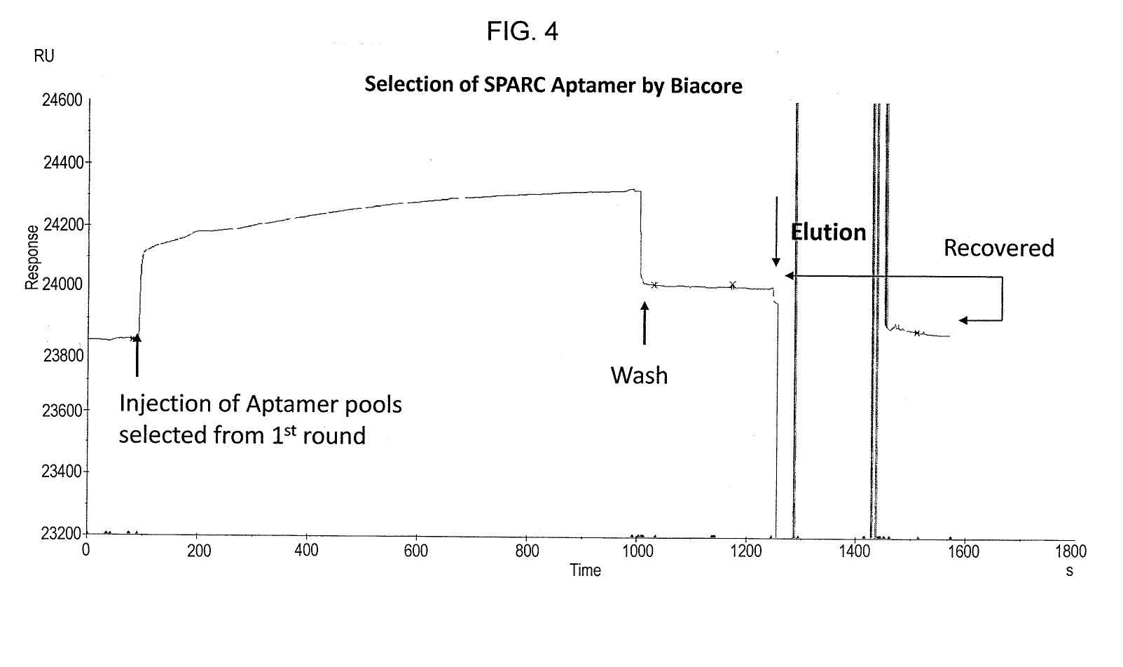

A library of single stranded DNA oligonucleotides, representing approximately 3×10¹⁵ DNA sequences, was synthesized by Invitrogen (Carlsbad, Calif.). As in Example 1, each oligonucleotide in the library comprised approximately 30 random nucleotides flanked by 18 nt PCR primer sequenes. For the first selection cycle, this ssDNA library pool (5 nmol in 100 μL HBS binding buffer, 50 μM concentration) was denatured at 95° C. for 5 minutes, cooled to 4° C. for 1 minute, and folded at room temperature for 5 minutes. The ssDNA pool was injected for 2 minutes into one of SPARC immobilized flow cells at a flow rate of 5 μL/minute. On completion of the injection, the binding buffer was injected into the flow cell at a flow rate of 20 μL/min to briefly rinse the flow cell surface. Two μL of elution buffer (3 M NaCl, 10 mM EDTA) was then injected into flow cell and incubated for 2 minutes. A microrecovery sensogram of this process is provided in FIG. 4. The 2 μL of eluted aptamers were then deposited into a vial. The 1 μL of recovered DNA was used for PCR amplification for next round of selection.

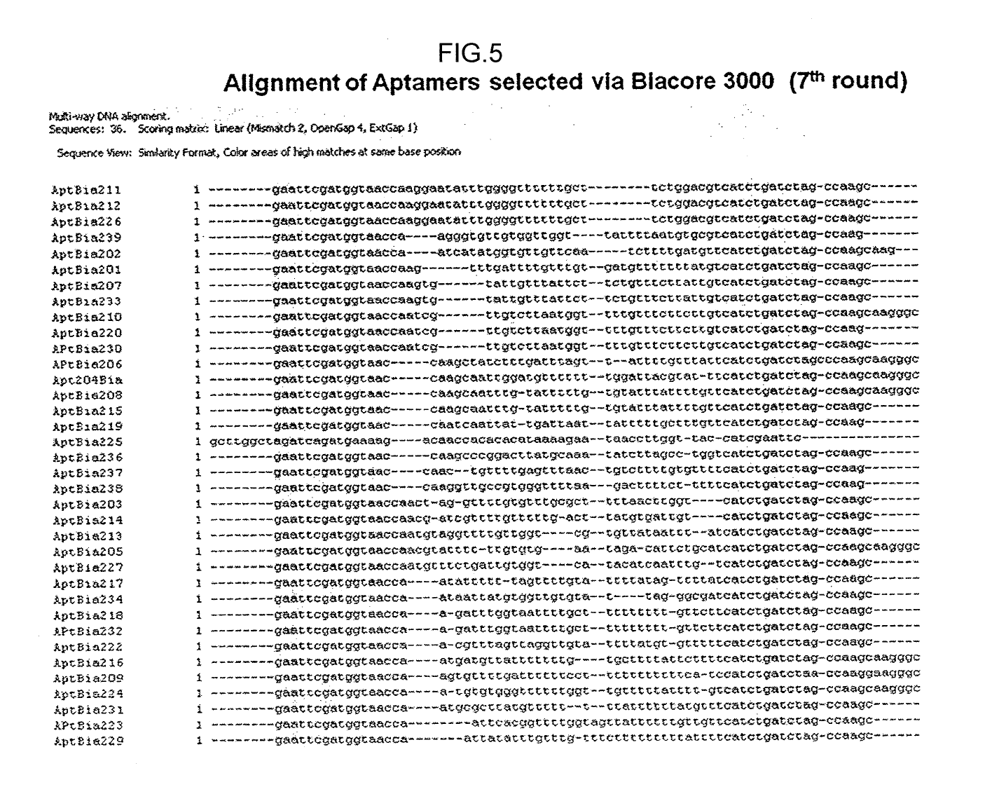

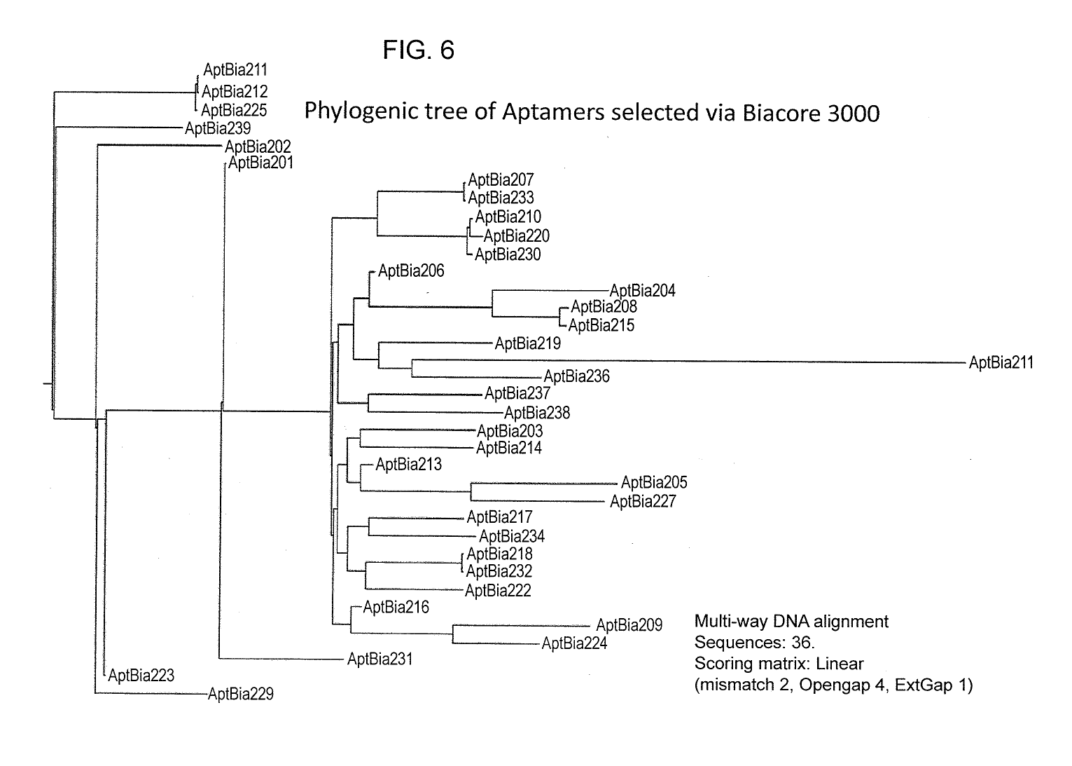

The protocol was repeated 7 times using the DNA amplified by PCR in each prior cycle. After the 7 cycles were completed, DNA sequences were analyzed and aligned using Clone Manager Professional Suite 8. An alignment of 36 sequences from round 7 is provided in FIG. 5. A phylogenic tree (FIG. 6) graphically illustrates similarity among sequences.

These results provide additional SPARC binding oligonucleotides having a range of homology at the primary structural level.

### Example 3

This example demonstrates the properties of aptamer-SPARC binding kinetics using surface plasmon resonance (SPR) technology.

20 μM each of biotinylated Apt180 (SEQ ID NO: 1), biotinylated Apt187 (SEQ ID NO: 2), or a control scrambled aptamer with biotin coupled to the 3′-end were immobilized onto commercially prepared SA sensor chips (GE/Biacore International AB), which have preimmobilized streptavidin, to allow biotin capture. Typically 1200 to 1300 RU of biotinylated aptamers were immobilized.

Several different concentrations of Biol SPARC (0.625 μM, 1.25 μM, 2.5 μM, 5 μM, and 10 μM) were injected over the sensor chips at a flow rate of 30 μL/min for 2 minutes for ka measurements. For each concentration, KD was assessed during the approximately 5-10 minute dissociation stage. The chip was regenerated by a 50 second injection of 1 M NaCl, 3.3 mM EDTA at a flow rate of 30 μL/min. Response curves were generated by subtracting the response of the reference cell from that of the experimental cell and the data were analyzed using in the BIACORE 3000® control software. Binding kinetics were calculated by simultaneously fitting the KA and KD data to a 1:1 Langmuir binding model (unless a different binding model was indicated).

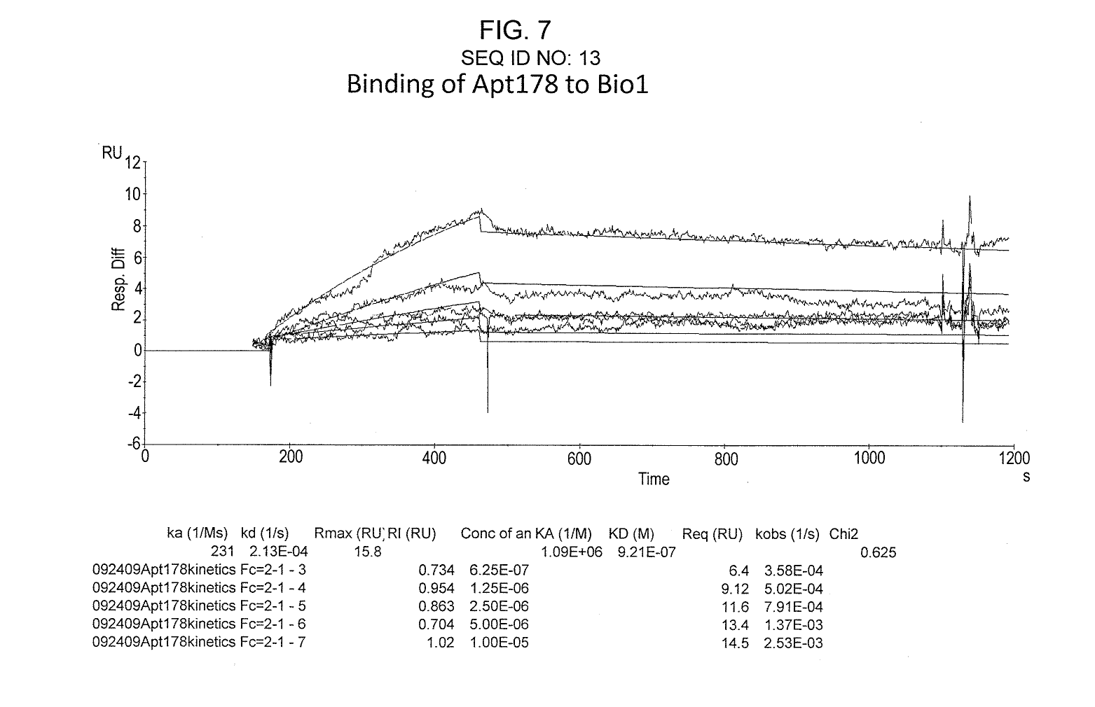

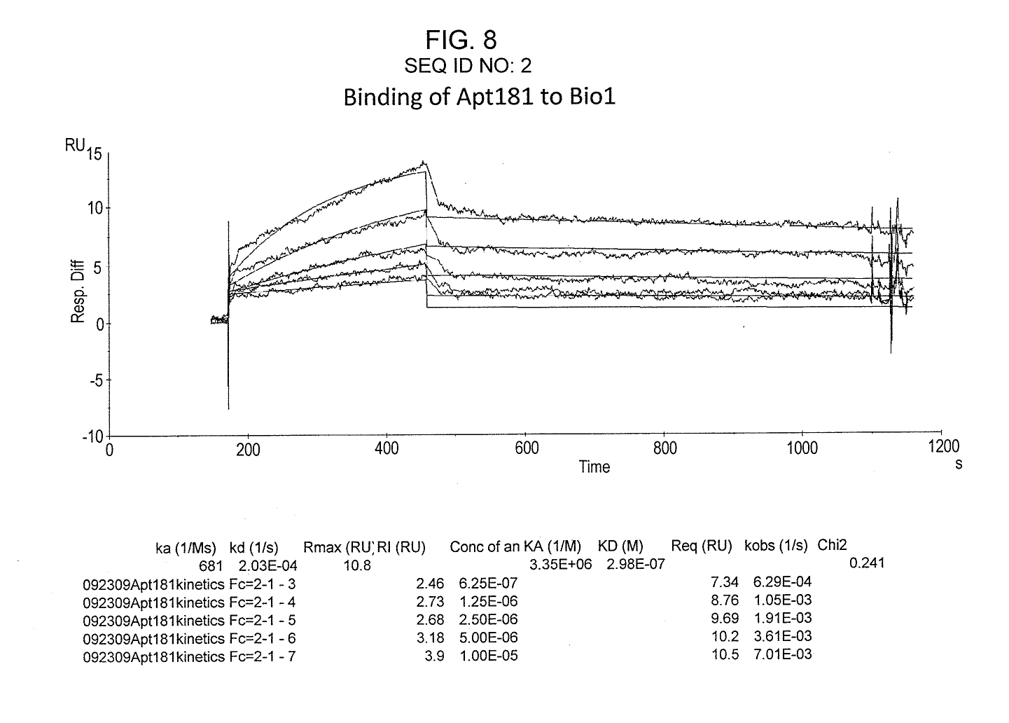

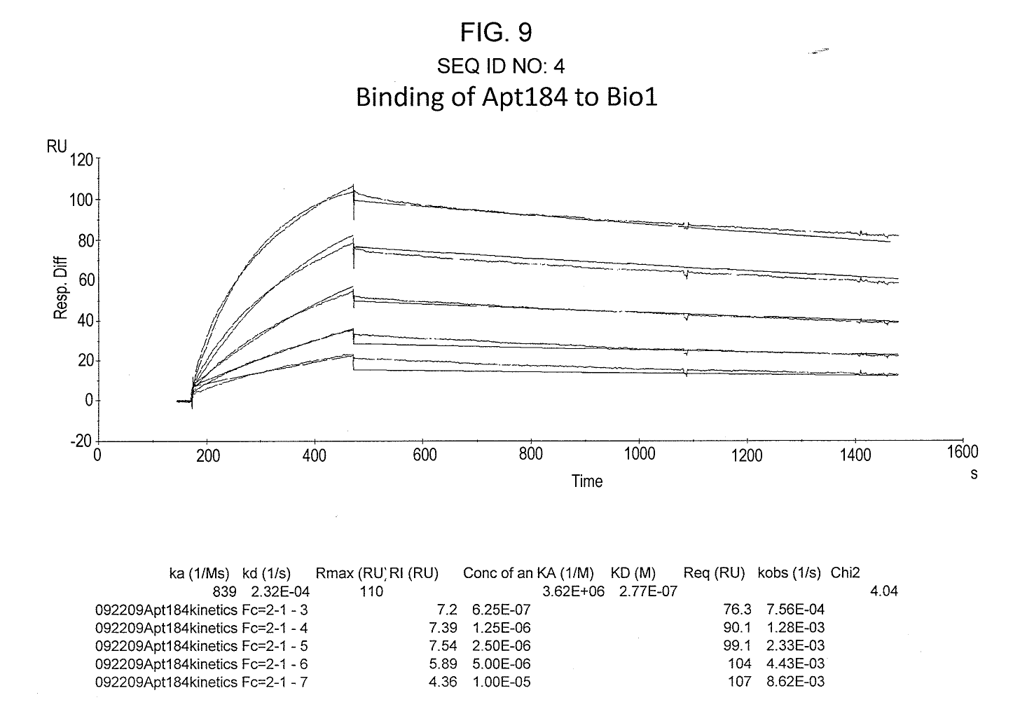

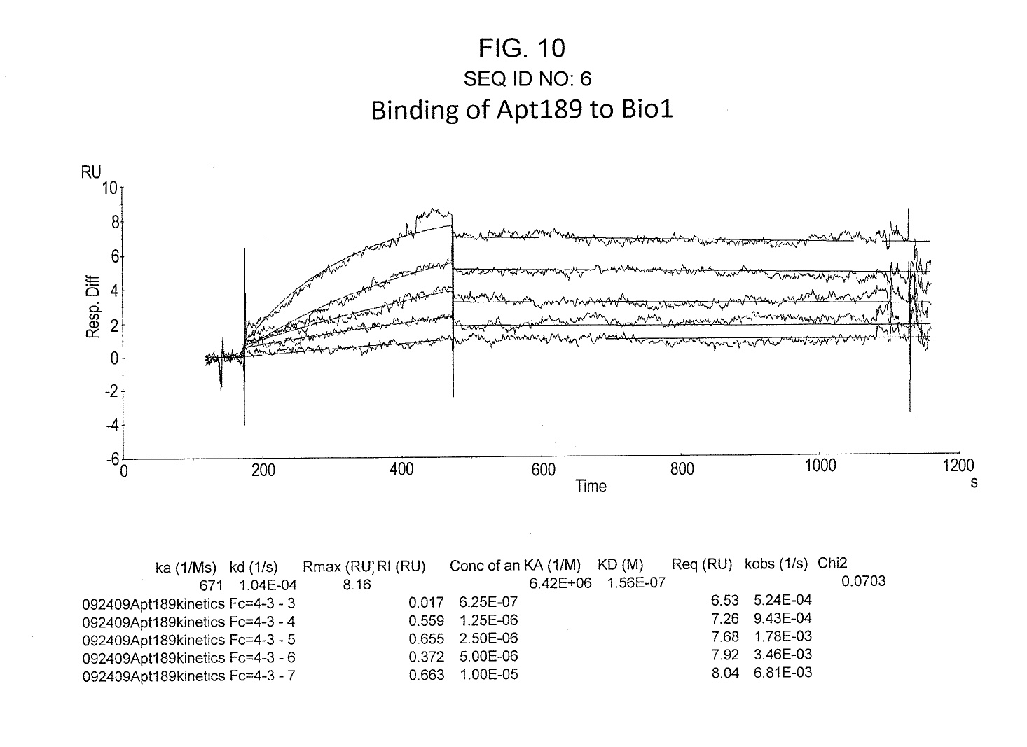

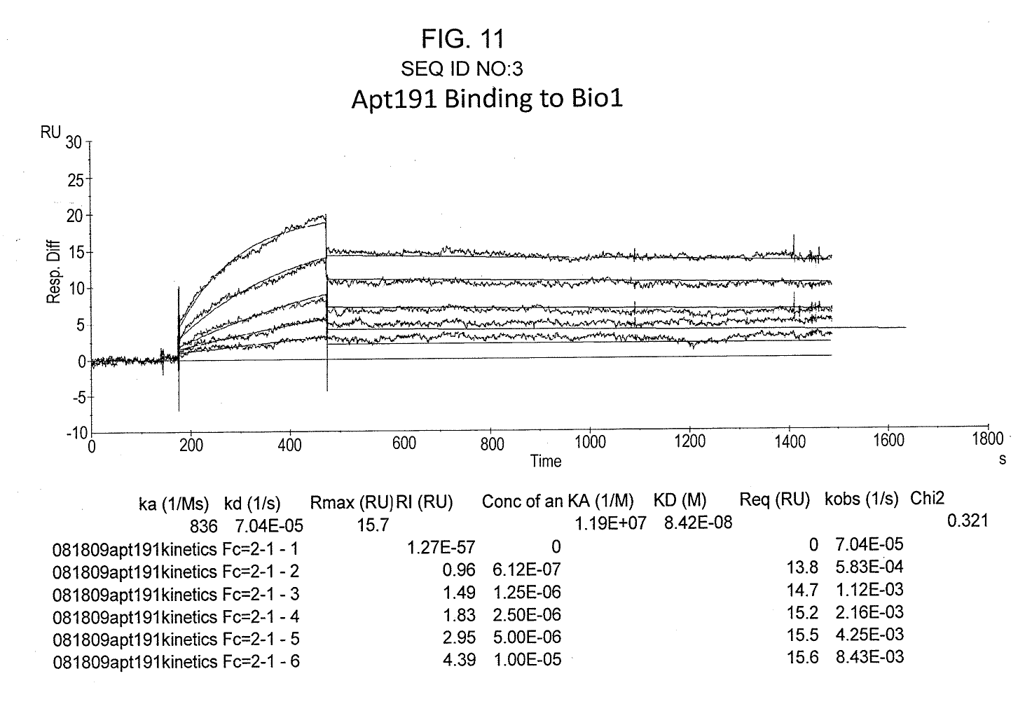

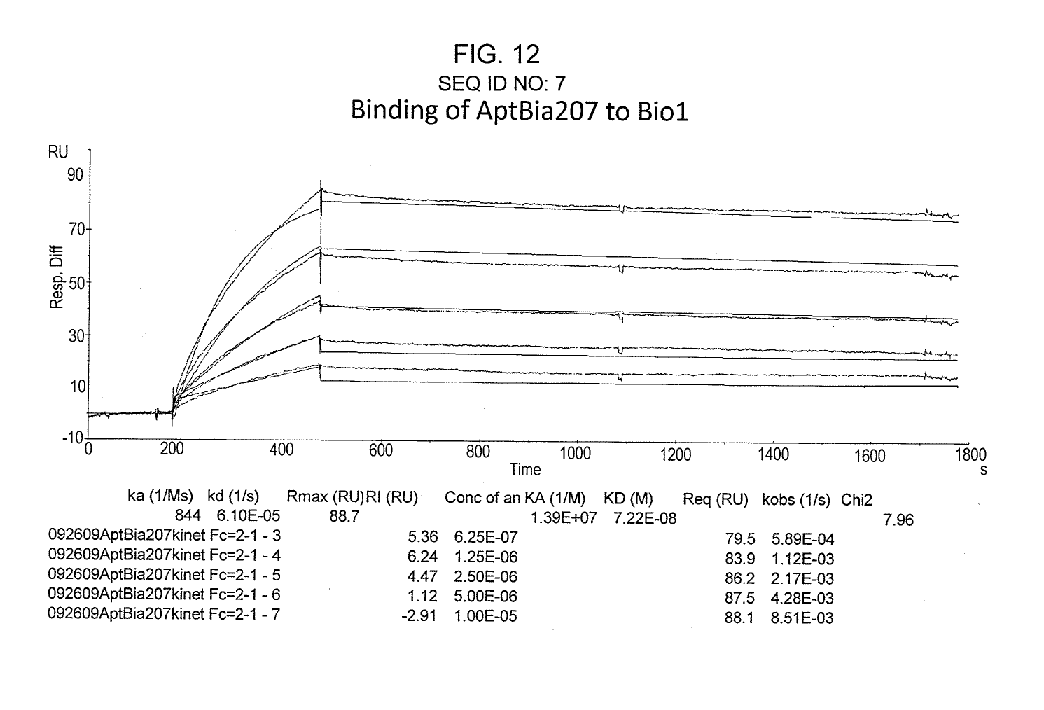

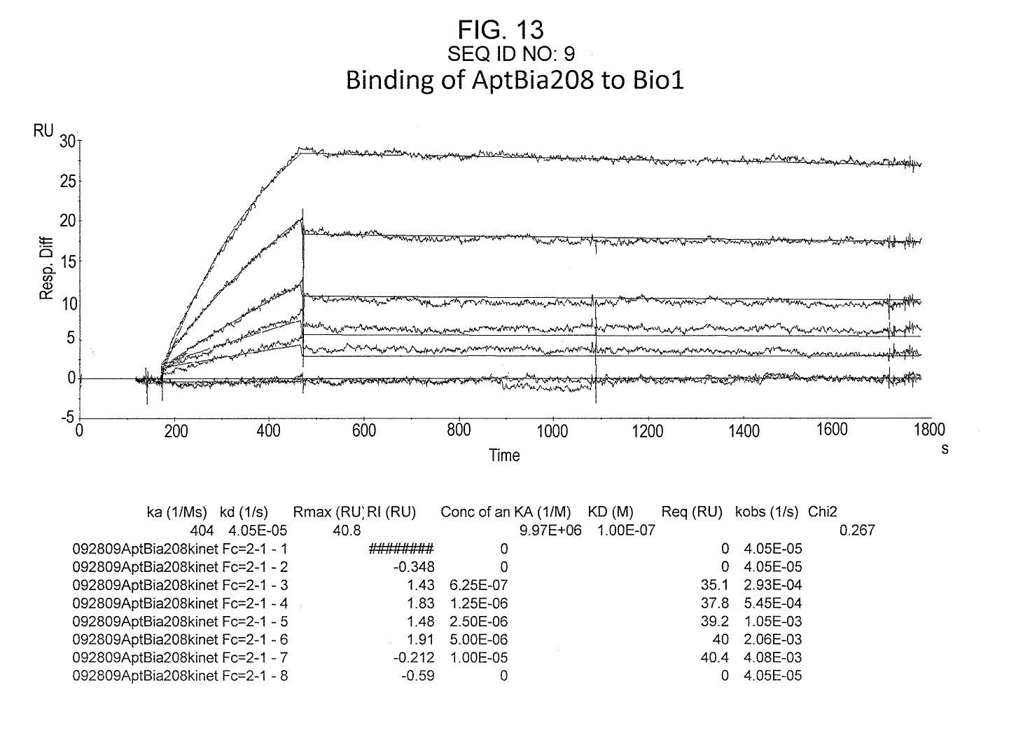

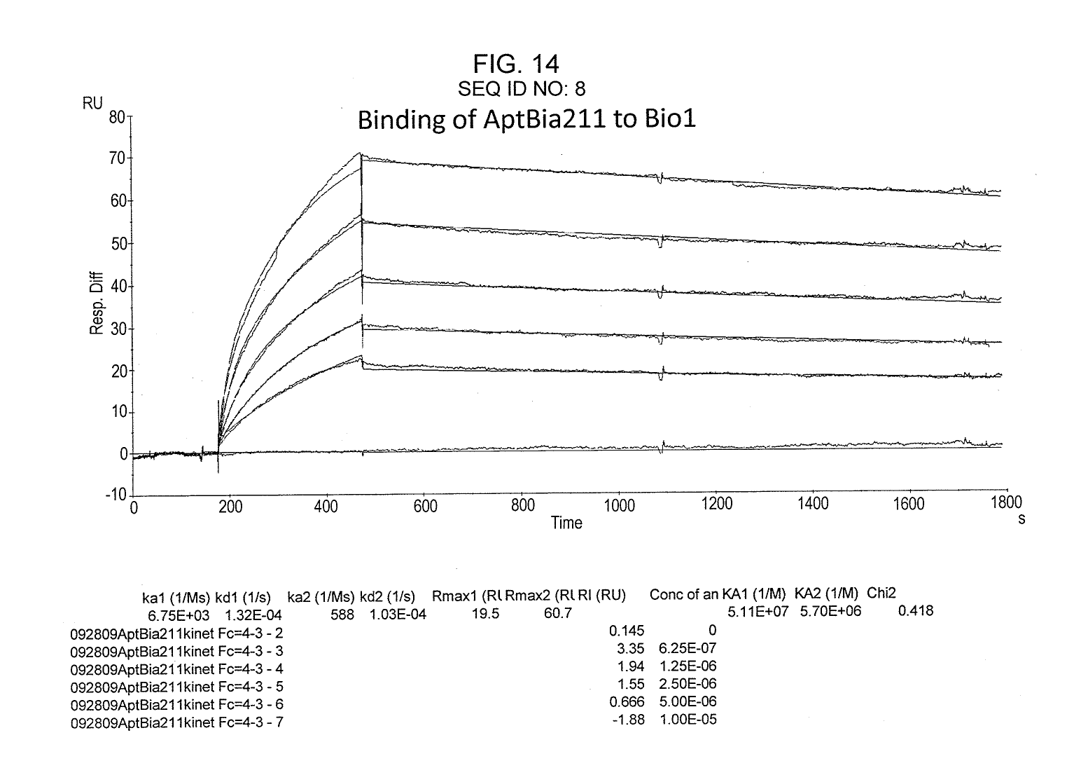

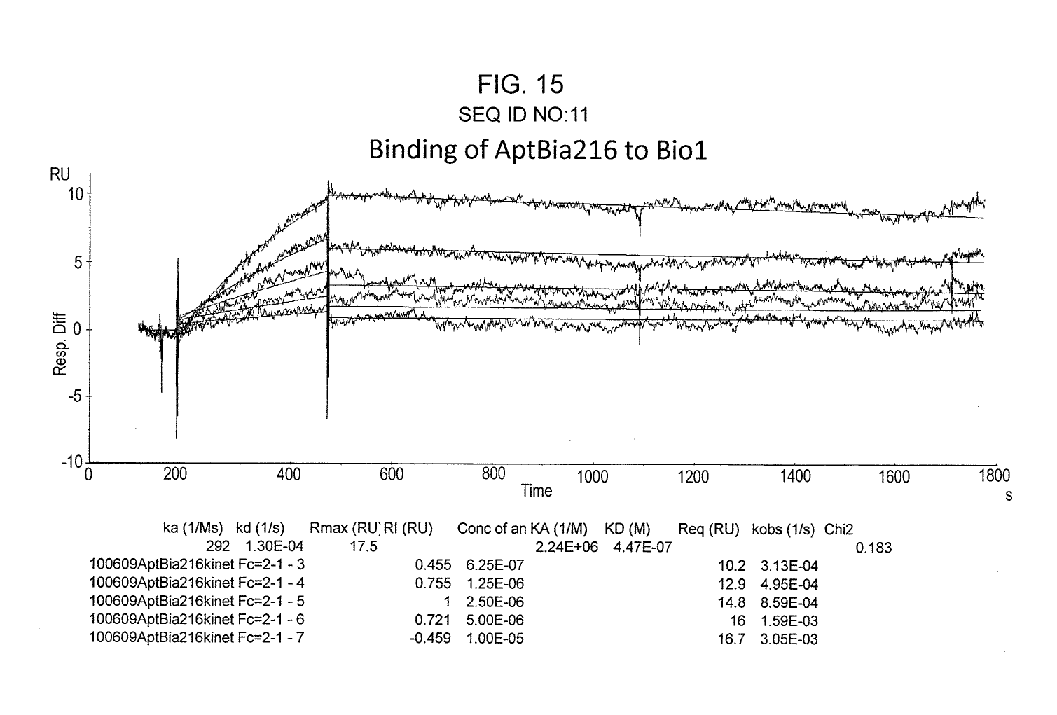

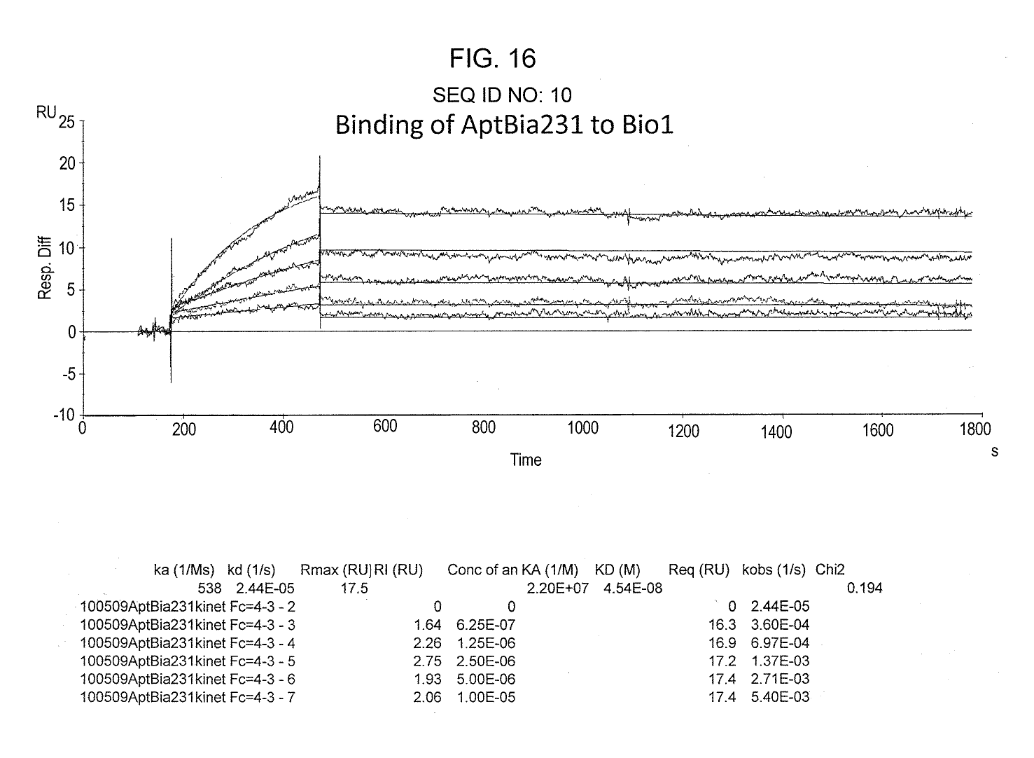

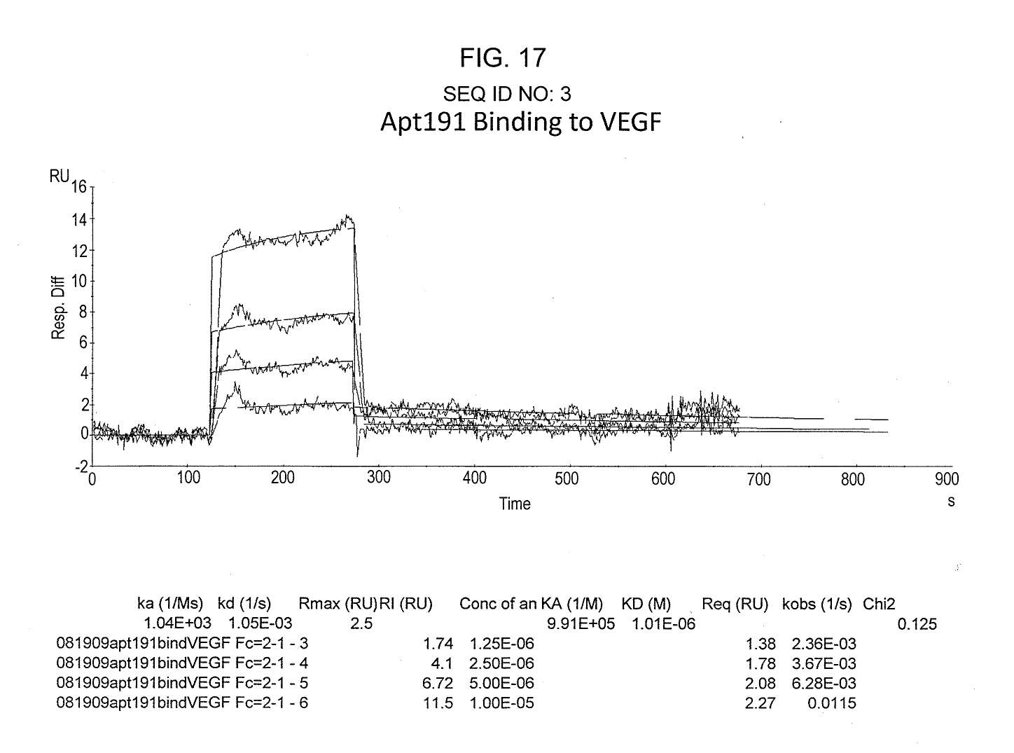

A concentration series of selected oligonucleotide DNA were injected over both reference surface and SPARC surface. Reference surfaces were used to subtract bulk refractive index responses from the specific SPARC binding signal as well as to ensure that there is no nonspecific interaction with the sensor chip surface. Exemplary sensorgrams depicting binding and dissociation of Apt178 (KD 0.921 μM), Apt181 (SEQ ID NO: 5; KD 0.298 μM), Apt184 (SEQ ID NO: 4; KD 0.277 μM), Apt189 (SEQ ID NO: 6; 72.2 nM), Apt191 (SEQ ID NO: 3; KD 84.2 nM), Apt207 (SEQ ID NO: 7; KD 0.139 μM), Apt208 (SEQ ID NO: 9; KD 0.100 μM), Apt211 (SEQ ID NO: 8; fitted to a heterogenous ligand model, with KD1 0.132 mM and KD2 0.103 mM), Apt216 (SEQ ID NO: 11; KD 0.447 μM), and Apt231 (SEQ ID NO: 10; KD 45.4 nM) are provided at FIGS. 7-16, respectively. The injection of SPARC aptamer Apt191 over a VegF immobilized sensorchip demonstrated no specific interaction between the aptamer and VegF (FIG. 17).

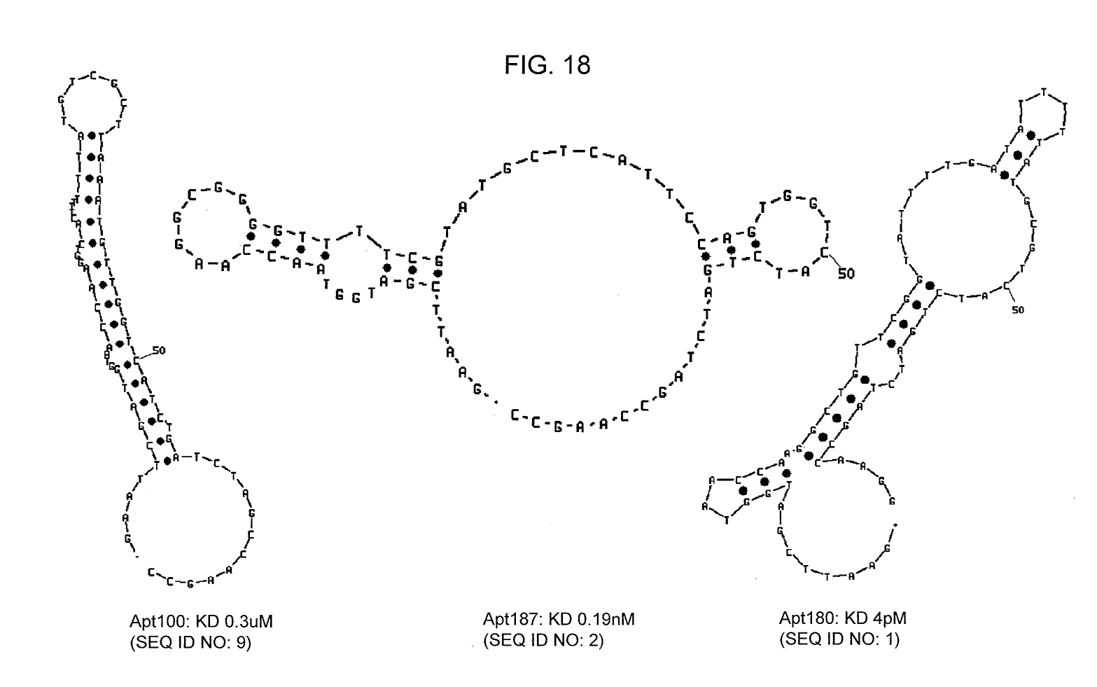

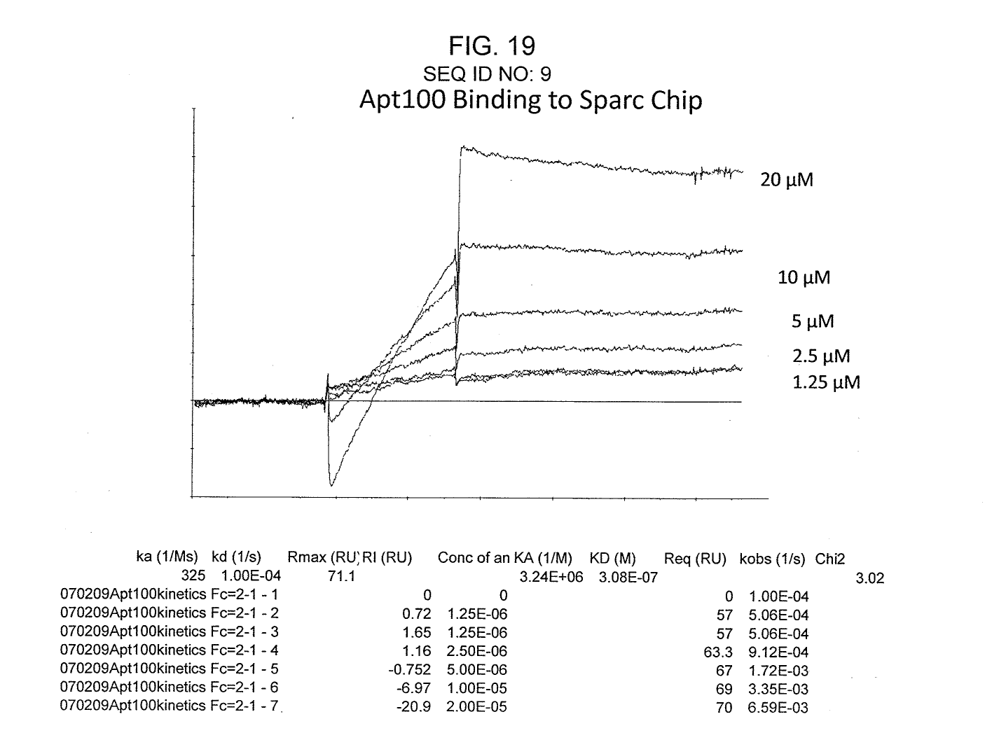

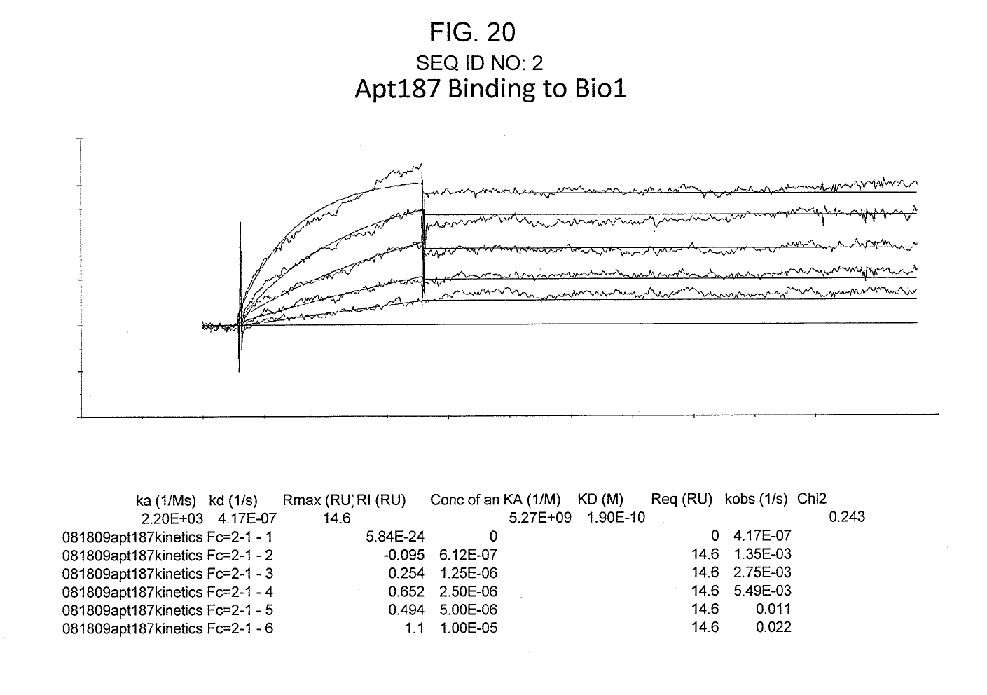

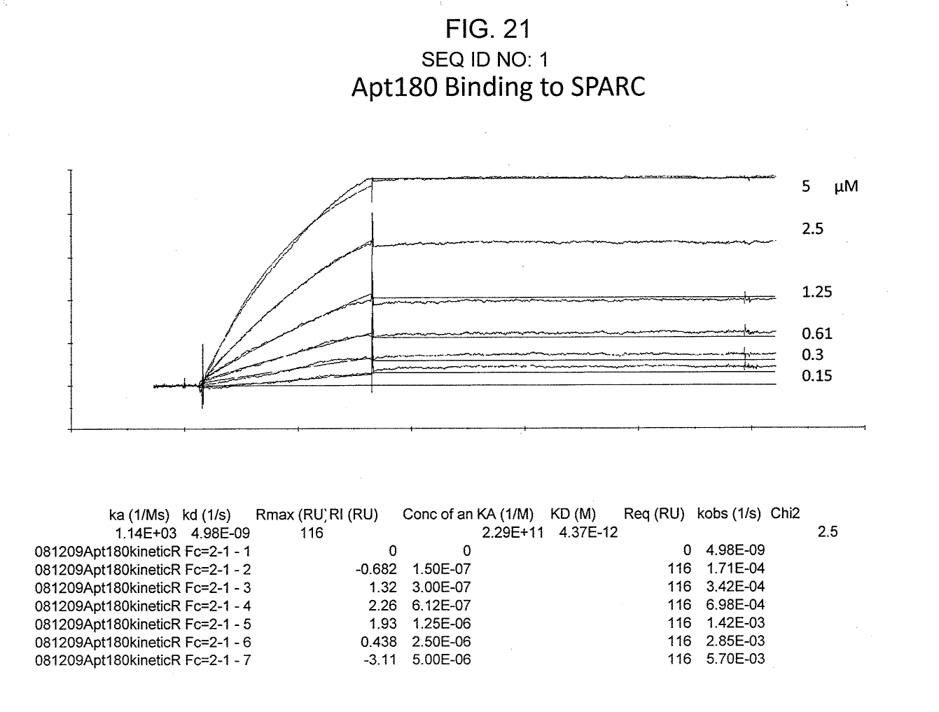

Using Clone Manager Professional Suite 8, secondary structures of at least three aptamers (Apt 100, Apt187, and Apt180) were modeled as shown in FIG. 18. For the aptamers evaluated, complexity of the secondary structure increased as binding affinity increased. That is, Apt100 (SEQ ID NO: 12), having KD of approximately 0.3 μM (FIG. 19), has a less complex structure than Apt187 (FIG. 20), which has KD 0.19 nM, which in turn has a less complex structure than Apt180, which has a KD of approximately 4 pM (FIG. 21).

These results show that certain of the identified aptamers are highly specific for SPARC and at least one, Apt180 (SEQ ID NO: 1) has picomolar binding specificity.

### Example 4

This example demonstrates various properties of two SPARC binding aptamers, Apt180 (SEQ ID NO: 1) and Apt 187(SEQ ID NO:2), two aptamers selected using the GST sepharose affinity chromatography methodology described in Example 1.

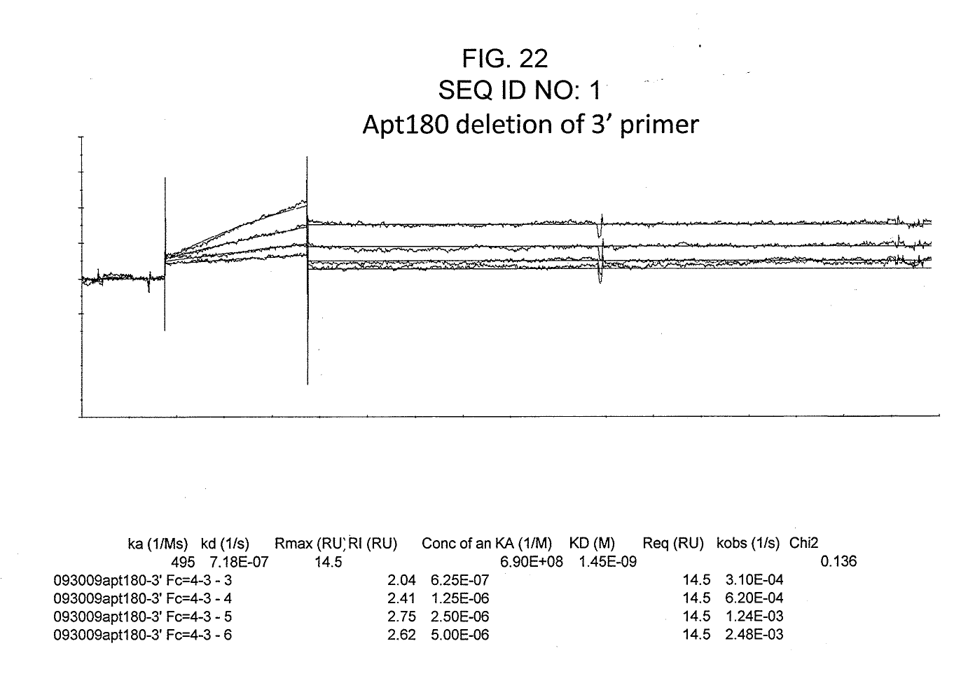

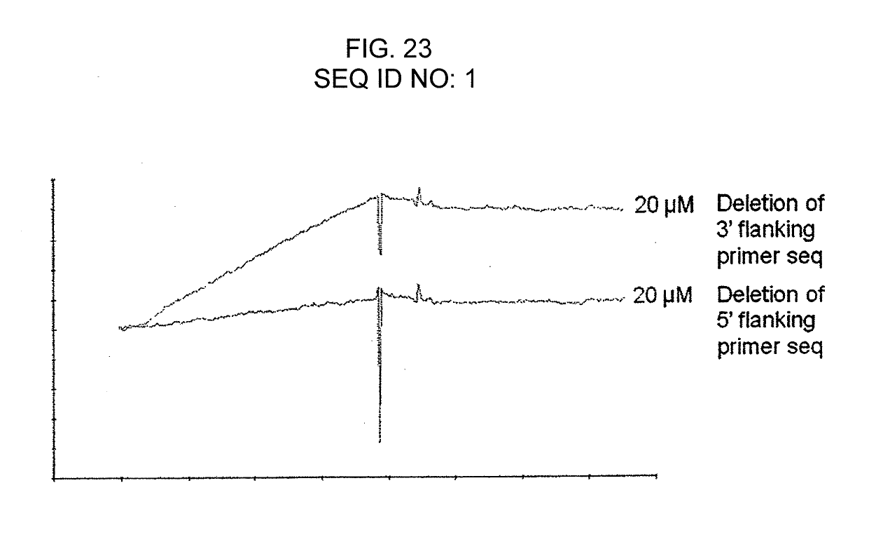

FIG. 21 depicts a sensorgram showing the binding and dissociation of Apt180 (SEQ ID NO: 1) to a Biol-SPARC immobilized CM5 sensorchip as described in Example 3. K_{D} under these conditions was calculated at 4.4 pM. Apt180 was then modified to determine the importance, if any, of the 5′ and 3′ primer regions of Apt180 to Apt180's SPARC binding affinity. FIG. 22 is a sensorgram showing the binding and dissociation of Biol-SPARC and a modified Apt180 having the 3′ primer region of the aptamer removed. As described in Example 3, Biol-SPARC was immobilized to CM5 sensorchip and various concentrations (0.625 μM, 1.25 μM, 2.5 μM, 5 μM, and 10 μM) of the 3′ primer deleted Apt180, were injected over the Biol-SPARC sensor chip. The resulting K_{D} calculated from this experiment was 1.45 nM. Similarly, another modified Apt180 aptamer was prepared having the 5′ primer region deleted. Binding and dissociation of Biol-SPARC to the two modified versions of Apt180 are further illustrated in FIG. 23. The sensorgram shows that the deletion of the 5′ primer region reduced Apt180 (SEQ ID NO: 1) binding to SPARC an did so by a greater margin than did the deletion of the 3′ primer region.

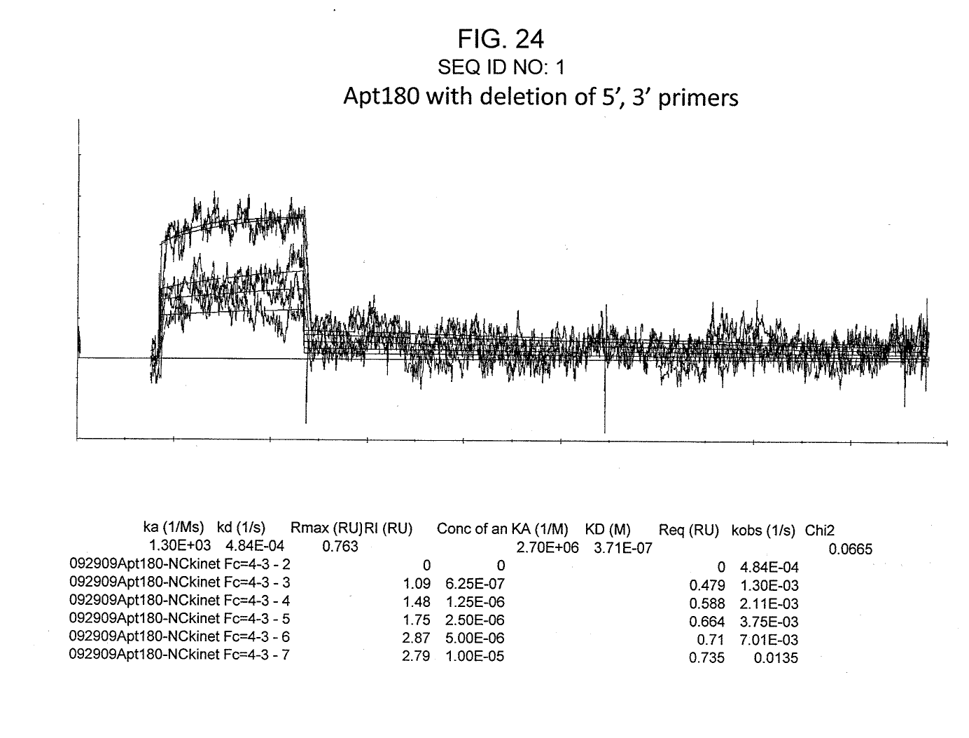

FIG. 24 is a sensorgram showing the binding and dissociation of Biol-SPARC and a modified Apt180 wherein both the 5′ and 3′ primer regions of the aptamer are deleted. As before, various concentrations of the modified Apt180 were injected over a Biol-SPARC immobilized sensorchip. Deletion of both the 5′ and 3′ primer regions further decreased binding affinity over the unmodified Apt180 or the 3′ deletion of Apt180.

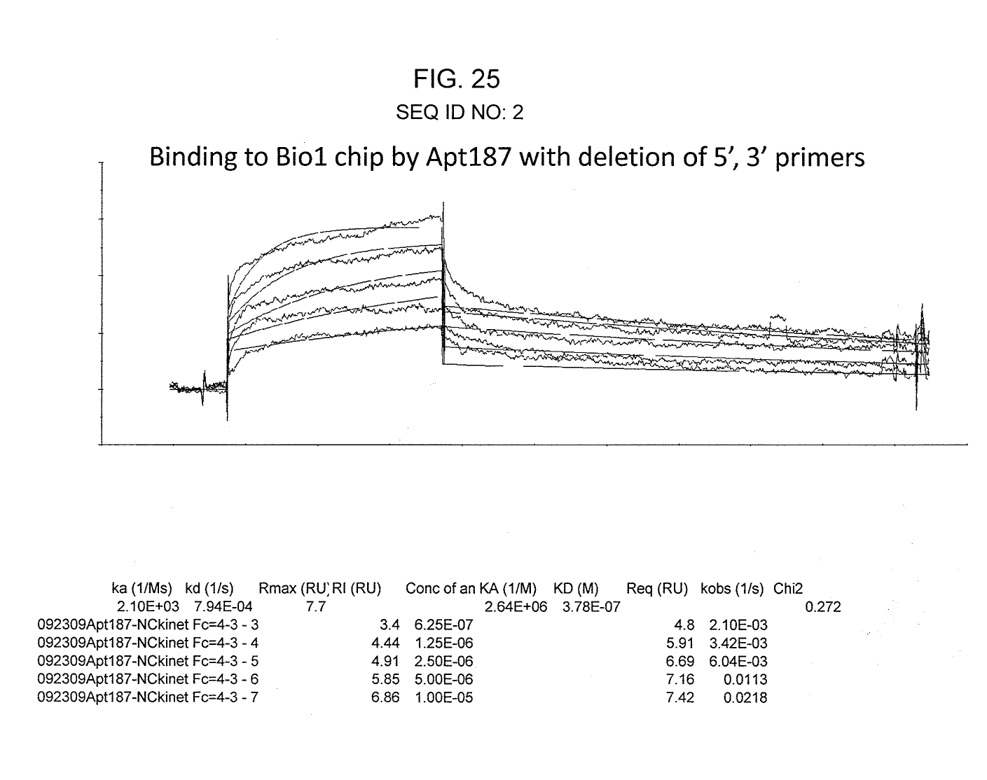

Similar deletions of the 5′ and 3′ primer regions of Apt187 likewise decreased binding affinity of Apt187 (FIG. 25).

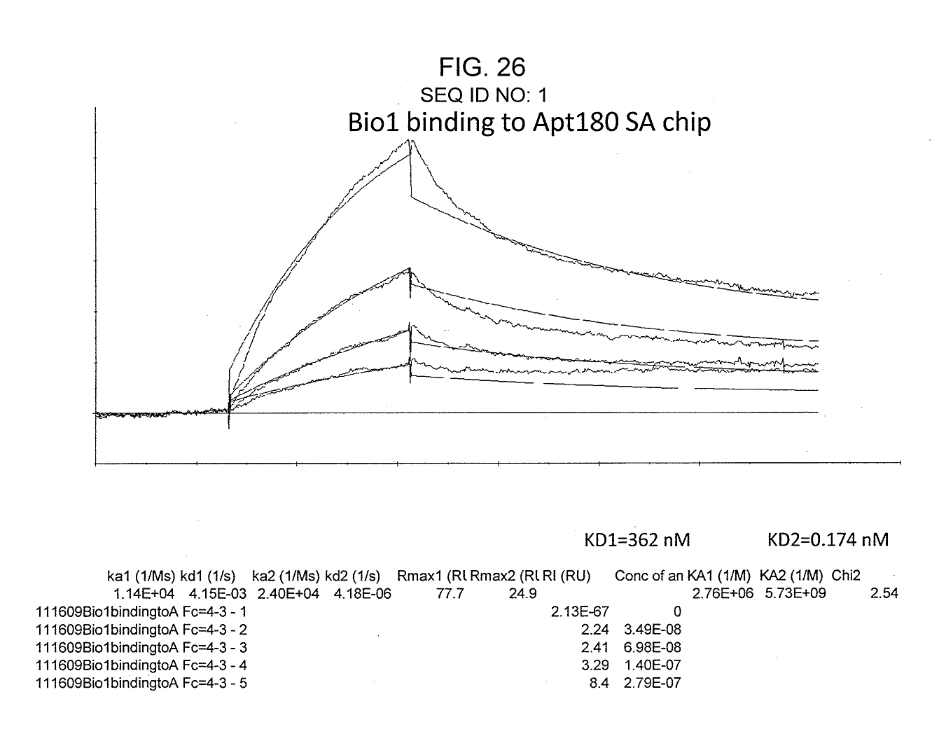

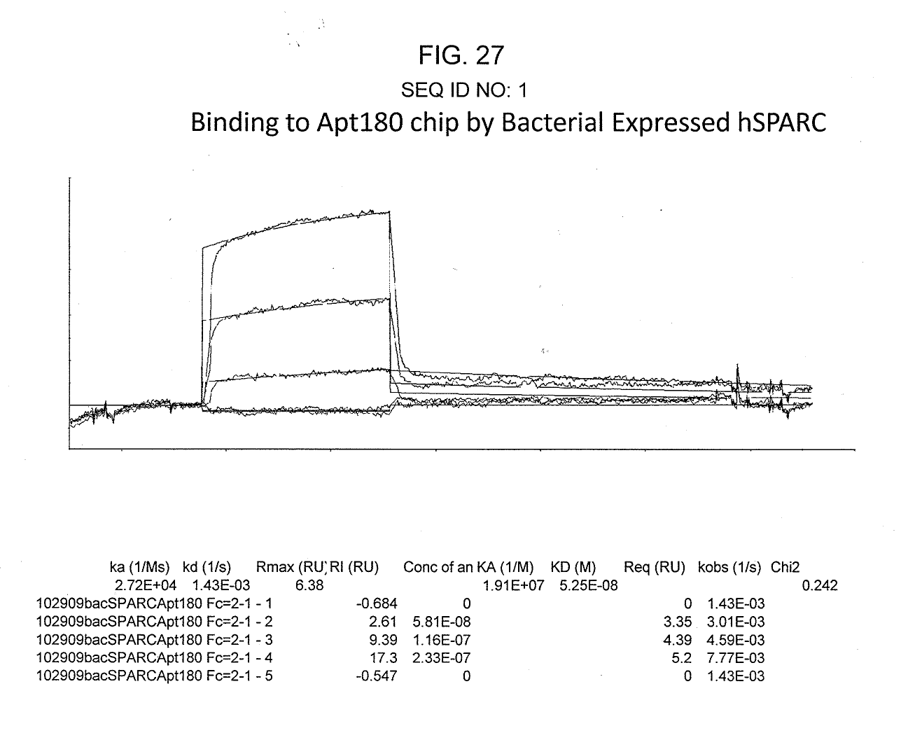

Using methods as described in Examples 2-3, above, biotinylated Apt180 and Apt187 were immobilized on CM5 sensorchips immobilized with streptavidin (SA sensorchips). Biol-SPARC was injected at concentrations ranging from 34.9 nM to 840 nM over the immobilized Apt180 and Apt187. For Apt180, the resulting curves were best fitted by heterogeneous ligand model indicating the possible presence of multiple binding sites. K_{D1} was calculated at 362 nM, and K_{D2} was 0.174 nM (FIG. 26). For Apt187, a 1:1 Langmuir model was appropriate, and a K_{D} of 30.2 nM was calculated (FIG. 27).

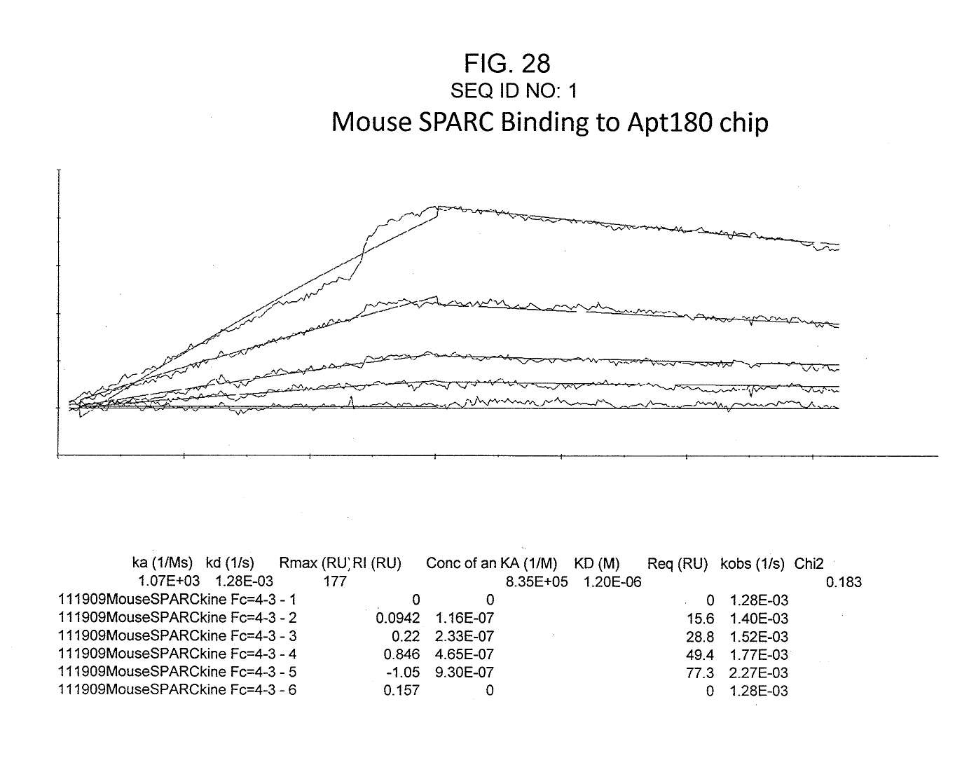

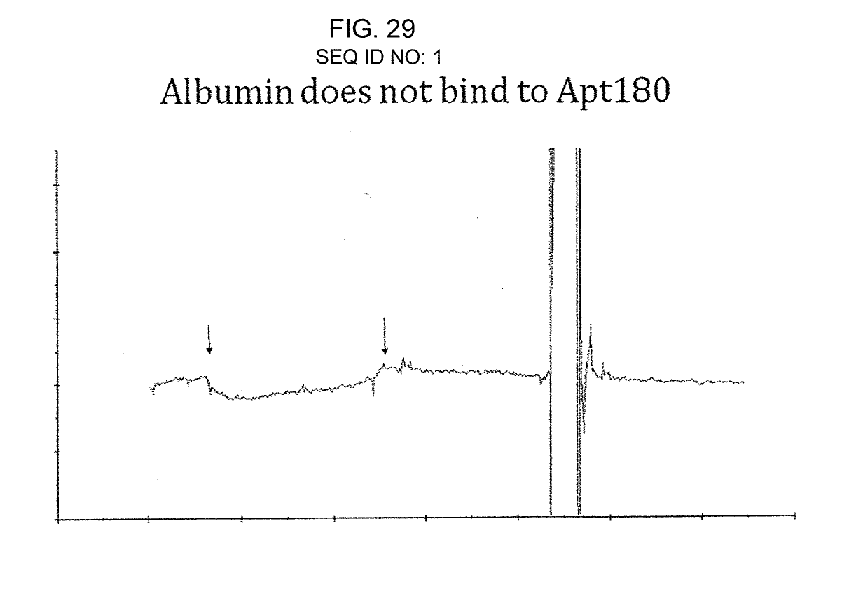

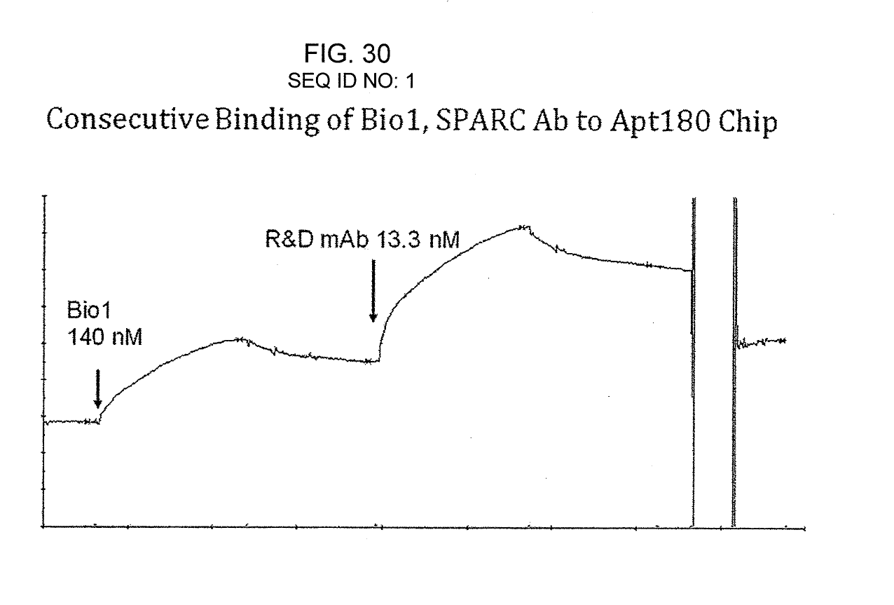

Binding of other SPARC homologs to immobilized Apt180 was also assayed: E. coli-expressed hSPARC, at a K_{D} of 52.5 nM (FIG. 28); and mouse SPARC purified from PYS-2 cells (parietal yolk Sac) (Sigma), at a K_{D} of 1.20 μM (FIG. 29). FIG. 30 depicts a sensorgram for an additional assay indicating that there is negligible binding between Apt180 (SEQ ID NO: 1) and bovine serum albumin (BSA). To further demonstrate the specificity of Apt180 (SEQ ID NO: 1) for human SPARC, a binding assay was also performed which shows that Apt 180 (SEQ ID NO: 1) binds negligibly to Vascular Endothelial Growth Factor (VEGF).

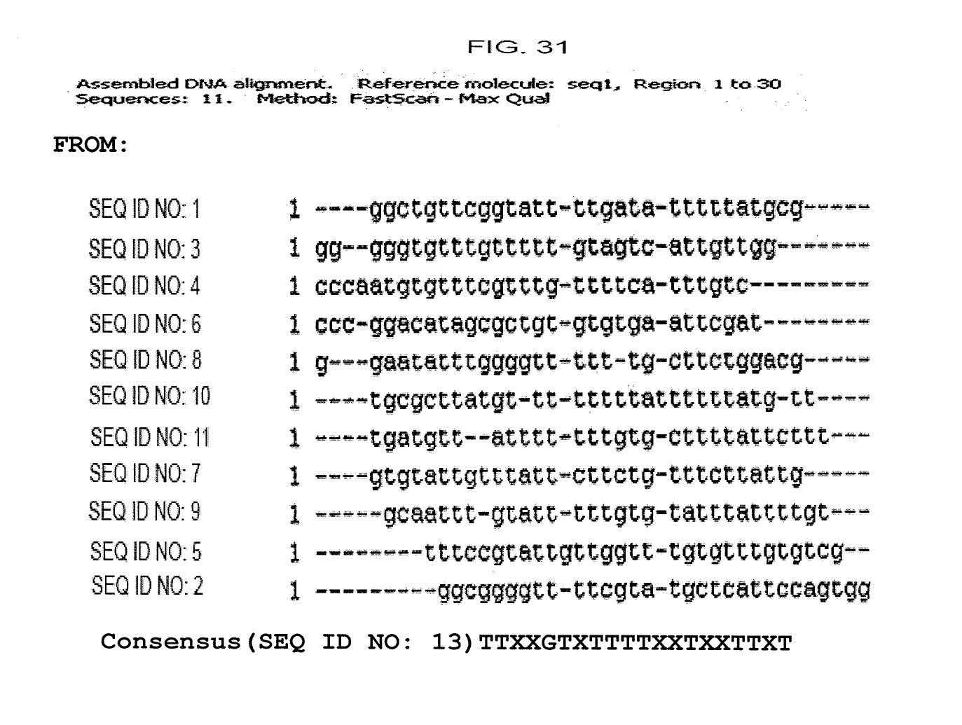

FIG. 31 depicts the consecutive binding of Biol-SPARC and anti-SPARC antibody to Apt 180. 3′ biotinylated Apt180 (SEQ ID NO: 1) was immobilized to CM5 SA (streptavidin) chip. 140 nM of Biol was injected over Apt180 (SEQ ID NO: 1) SA chip, followed by injection of 13.3 nM of a commercially available anti-SPARC monoclonal antibody. The results show that the monoclonal anti-SPARC antibody bound to the SPARC protein captured by Apt180, and confirms that Apt180 binds to SPARC with specificity.

### Example 5

This example illustrates the use of the aptamers of the present invention to prepare a consensus sequence.

Eleven SPARC binding aptamers having high affinity for SPARC, produced as described in Example 1 or 2, were sequenced and aligned using Clone Manager Professional Suite 8 (see FIG. 31). This analytical approach arrived at a consensus sequence of TTxxGTxTTTTxxTxxTTxT (SEQ ID NO: 14).

The consensus sequence of nucleic acids prepared by these analyses can be used to identify additional aptamers capable of binding SPARC.

### Example 6

This example illustrates the preparation of SPARC-binding aptamers having stability enhancing features.

A DNA or RNA library is commercially prepared and SPARC-binding aptamers are obtained therefrom using the methods of Examples 1 and/or 2, above. Resulting aptamers are modified using conventional techniques to introduce one or more stability enhancing features such as, e.g., the substitution of DNA or RNA residues with LNA residues.

After modification, surface plasmon resonance is used to confirm that SPARC binding is maintained at a suitable level.

### Example 7

This example illustrates the use of the aptamers of the present invention to diagnose a proliferative disease.

A suitable quantity of Apt180 is synthesized via chemical means known in the art. The aptamers are conjugated to a diagnostic agent suitable for medical imaging, such as a radionuclide, using a conjugation method known in the art.

The composition is applied to tissue samples taken from a test cohort of patients suffering from a proliferative disease associated with the overexpression of SPARC, e.g. breast cancer. The composition is likewise applied to tissue samples taken from a negative control cohort, not suffering from a proliferative disease.

The use of appropriate medical imaging techniques on the test cohort samples indicates the presence of disease, while the same techniques applied to the control cohort samples indicate the absence of disease.

The results will show that the aptamers of the present invention are useful in diagnosing proliferative diseases.

### Example 8

This example illustrates the use of the aptamers of the present invention to treat a proliferative disease in mice.

A suitable quantity of Apt180 is synthesized via chemical means known in the art. The aptamers are conjugated to a chemotherapeutic agent, such as Doxil, using a conjugation method known in the art. The conjugate is formulated in an aqueous composition.

The composition is administered intravenously, in one or more doses, to a test cohort of mice suffering from a proliferative disease associated with the overexpression of SPARC, e.g. a breast cancer model. A control cohort, not suffering from a proliferative disease is administered the identical composition intravenously, according to a corresponding dosage regimen.

Pathological analysis of tumor samples and/or mouse survival indicate that mortality and/or morbidity are improved in the test cohort over the control cohort.

The results will show that the antibodies of the present invention are useful in treating proliferative diseases.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising a SPARC binding aptamer wherein the SPARC binding aptamer comprises a nucleic acid of SEQ ID NOs: 1-11, or a modification or homolog thereof.

2. The composition of claim 1, further comprising an active agent, wherein the active agent is conjugated to the SPARC binding aptamer.

3. The composition of claim 2, wherein the active agent comprises a therapeutic agent or a diagnostic agent.

4. The composition of claim 3, wherein the composition, when administered to an animal, results in an enhancement of the delivery of the active agent to a disease site relative to delivery of the active agent alone.

5. The composition of claim 3, wherein the therapeutic agent or diagnostic agent is a therapeutic agent selected from the group consisting of tyrosine kinase inhibitors, kinase inhibitors, biologically active agents, biological molecules, radionuclides, adriamycin, ansamycin antibiotics, asparaginase, bleomycin, busulphan, cisplatin, carboplatin, carmustine, capecotabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, melphalan, methotrexate, rapamycin (sirolimus), mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, anti-vascular endothelial growth factor compounds (“anti-VEGFs”), anti-epidermal growth factor receptor compounds (“anti-EGFRs”), 5-fluorouracil and derivatives, radionuclides, polypeptide toxins, apoptosis inducers, therapy sensitizers, enzyme or active fragment thereof, and combinations thereof.

6. The composition of claim 3, wherein the therapeutic agent or diagnostic agent is a therapeutic agent comprising an antibody or antibody fragment.

7. The composition of claim 6, wherein the antibody or antibody fragment is an Fc fragment of IgG, or IgA, or IgD, or IgE, or IgM.

8. The composition of claim 6, wherein the antibody or antibody fragment mediates one or more of complement activation, cell mediated cytotoxicity, or opsonization.

9. The composition of claim 3, wherein the therapeutic agent or diagnostic agent is a diagnostic agent selected from the group consisting of radioactive agents, MRI contrast agents, X-ray contrast agents, ultrasound contrast agents, and PET contrast agents.

10. The composition of claim 1, wherein the composition comprises a liposome.

11. The composition of claim 1, wherein the composition comprises an albumin nanoparticle.

12. The composition of claim 1, wherein the SPARC binding aptamer comprises a nucleic acid of SEQ ID NO: 1 or SEQ ID NO: 2.

13. The composition of claim 1, wherein the SPARC binding aptamer comprises a nucleic acid of SEQ ID NO: 1.

14. The composition of claim 1, wherein the SPARC binding aptamer is a DNA molecule.

15. The composition of claim 1, wherein the composition further comprises a pharmaceutically acceptable carrier.

16. The composition of claim 1, wherein the composition is administered to a patient intravenously, intramuscularly, subcutaneously, intraperitoneally, topically, via inhalation, intranasally, rectally or orally.

17. A method for diagnosing or treating a disease in a mammal comprising: administering a diagnostically or therapeutically effective amount of a composition comprising a SPARC binding aptamer, wherein the SPARC binding aptamer comprises a nucleic acid of SEQ ID NOs: 1-11, or a modification or homolog thereof.

18. The method of claim 18, wherein the composition further comprises an active agent conjugated to the SPARC binding aptamer

19. The method of claim 19, wherein the active agent comprises a therapeutic agent or a diagnostic agent.

20. The method of claim 19, wherein administering a therapeutically effective amount of the composition to the mammal results in:
(a) an enhancement of the delivery of the active agent to a disease site relative to delivery of the active agent alone, or
(b) an enhancement of SPARC clearance resulting in a decrease in blood level of SPARC—preferably 50%, or 25%, or 10%.

21. The method of claim 20, wherein the therapeutic agent or diagnostic agent is a therapeutic agent selected from the group consisting of tyrosine kinase inhibitors, kinase inhibitors, biologically active agents, biological molecules, radionuclides, adriamycin, ansamycin antibiotics, asparaginase, bleomycin, busulphan, cisplatin, carboplatin, carmustine, capecotabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, melphalan, methotrexate, rapamycin (sirolimus), mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, transplatinum, anti-vascular endothelial growth factor compounds (“anti-VEGFs”), anti-epidermal growth factor receptor compounds (“anti-EGFRs”), 5-fluorouracil and derivatives, radionuclides, polypeptide toxins, apoptosis inducers, therapy sensitizers, enzyme or active fragment thereof, and combinations thereof.

22. The method of claim 20, wherein the therapeutic agent or diagnostic agent is a therapeutic agent comprising an antibody or antibody fragment.

23. The method of claim 23, wherein the antibody or antibody fragment is an Fc fragment of IgG, or IgA, or IgD, or IgE, or IgM.

24. The method of claim 23, wherein the antibody or antibody fragment mediates one or more of complement activation, cell mediated cytotoxicity or opsonization.

25. The method of claim 21, wherein the therapeutic agent or diagnostic agent is a diagnostic agent selected from the group consisting of radioactive agents, MRI contrast agents, X-ray contrast agents, ultrasound contrast agents, and PET contrast agents.

26. The method of claim 18, wherein the composition further comprises a pharmaceutically acceptable carrier.

27. The method of claim 18, wherein the therapeutically effective amount of the composition is administered to a patient intravenously, intramuscularly, subcutaneously, intraperitoneally, topically, via inhalation, intranasally, rectally or orally.

28. The method of claim 18, wherein the SPARC binding aptamer comprises a nucleic acid of SEQ ID NO: 1.

29. The method of claim 18, wherein the SPARC binding aptamer comprises a nucleic acid of SEQ ID NO: 1 or SEQ ID NO: 2.

30. The method of claim 18, wherein the SPARC binding aptamer is a DNA molecule.

31. The method of claim 18, further comprising administering a therapeutically effect amount of albumin-bound nanoparticulate paclitaxel.

32. A method of diagnosing a disease or condition in an animal comprising:
(a) administering to the animal a diagnostically effective amount of a SPARC binding aptamer comprising SEQ ID NOs:1-11;
(b) detecting the amount of SPARC binding aptamer present in a particular site or tissue of the animal; and
(c) diagnosing that the disease or condition is present if the amount of SPARC binding aptamer present indicates that significantly greater than normal levels of SPARC are present in the particular site or tissue.

33. The method of claim 18, wherein the animal is human.

34. The method of claim 18, wherein the disease is a neoplastic, proliferative, or inflammatory disease, or a tissue-remodeling disease or disorder.

35. The method of claim 37, wherein the disease site is a tumor.

36. A kit for the detection or treatment of a disease comprising a pharmaceutical formulation and instructions for use of the formulation in the treatment of tumors, wherein the pharmaceutical formulation comprises a SPARC binding aptamer wherein the SPARC binding aptamer comprises a nucleic acid of SEQ ID NOs: 1-11, or 14 or modifications or homologs thereof.

37. The kit of claim 39 wherein the pharmaceutical formulation further comprises an active agent, wherein the active agent is conjugated to the SPARC binding aptamer, and wherein and the administration of the pharmaceutical formulation to an animal results in
(a) an enhancement of the delivery of the active agent to a disease site relative to delivery of the active agent alone or
(b) an enhancement of SPARC clearance resulting in a decrease in blood level of SPARC—preferably 50%, or 25%, or 10%.

38. A method for identifying a nucleic acid aptamer with high affinity for SPARC, the method comprising
(a) preparing a mixture of nucleic acids comprising random candidate nucleic acids;
(b) contacting the mixture of nucleic acids with a tagged SPARC peptide;
(c) partitioning the nucleic acids with greater affinity for SPARC from the mixture of nucleic acids;
(d) amplifying the nucleic acids of (c) to yield a mixture of candidate nucleic acids having increased affinity for SPARC relative to the mixture of (a);
(e) repeating steps (b) through (d) with the mixture of candidate nucleic acids amplified in (d);
(f) repeating steps (a) through (e) until a mixture of candidate nucleic acids having high affinity for SPARC is obtained; and
(g) identifying nucleic acid aptamers with high affinity for SPARC from the mixture of (f).

39. A method for identifying a nucleic acid aptamer with high affinity for SPARC, the method comprising
(a) preparing a sensor chip immobilized with SPARC using a surface plasmon resonance (SPR) instrument;
(b) preparing a mixture of nucleic acids comprising random candidate nucleic acids;
(c) injecting the mixture of nucleic acids onto a flow cell wherein the CM5 sensor chip is docked in the SPR instrument;
(d) partitioning the nucleic acids with greater affinity for SPARC from the mixture of nucleic acids by elution and recovery of bound nucleic acids;
e) amplifying the nucleic acids of (d) to yield a mixture of candidate nucleic acids having increased affinity for SPARC relative to the mixture of (b);
(f) repeating steps (c) through (e) with the mixture of candidate nucleic acids amplified in (e);
(g) repeating steps (c) through (f) until a mixture of candidate nucleic acids having high affinity for SPARC is obtained; and
(h) identifying nucleic acid aptamers with high affinity for SPARC from the mixture of (g).

40. A composition comprising a SPARC binding aptamer wherein the SPARC binding aptamer comprises a nucleic acid of the SPARC binding consensus sequence (SEQ ID NO: 14), or a modification or homolog thereof, wherein up to two of the specified bases of the SPARC binding consensus sequence are changed, and wherein binding affinity of the SPARC binding aptamer to SPARC, as measured by K_{d}, is between 10⁻⁶M and 10⁻⁹M.

41. The composition of claim 40 wherein the SPARC binding aptamer comprises uracil bases in place of thymidine bases

42. The composition of claim 40 wherein the SPARC binding aptamer comprises RNA.

43. A method for diagnosing or treating a disease in a mammal comprising: administering a diagnostically or therapeutically effective amount of a composition comprising a consensus SPARC binding aptamer wherein
(a) the consensus SPARC binding aptamer comprises a sequence of SEQ ID NO: 14, or a modification or homolog thereof, wherein up to two of the specified bases of the SPARC binding consensus sequence may be changed, and
(b) wherein binding affinity of the SPARC binding aptamer to SPARC, as measured by K_{d} is between 10⁻⁶M and 10⁻⁹M.

44. The method of claim 43, wherein the consensus SPARC binding aptamer comprises uracil bases in place of thymidine bases.

45. The method of claim 43, wherein the consensus SPARC binding aptamer comprises RNA.

46. A composition comprising a SPARC binding aptamer wherein the SPARC binding aptamer comprises a nucleic acid of SEQ ID NOs: 1-11, or a modification or homolog thereof, wherein up to two of the specified bases of the sequence are changed, and wherein binding affinity of the SPARC binding aptamer to SPARC, as measured by Kd, is between 10⁻⁶M and 10⁻⁹M.

47. A method for diagnosing or treating a disease in a mammal comprising: administering a diagnostically or therapeutically effective amount of a composition comprising a SPARC binding aptamer wherein
(a) the SPARC binding aptamer is a nucleic acid of SEQ ID NOs: 1-11, or a modification or homolog thereof, wherein up to two of the specified bases of the sequence may be changed, and
(b) wherein binding affinity of the SPARC binding aptamer to SPARC, as measured by Kd is between 10⁻⁶M and 10⁻⁹M.


SEQUENCE LISTING (15 sequences)

SEQ ID NO: 1 - DNA, 66 bp, Artificial Sequence
  Synthetic
gaattcgatggtaaccaaggctgttcggtattttgatatttttatgcgtcatctgatctagccaag

SEQ ID NO: 2 - DNA, 66 bp, Artificial Sequence
  Synthetic
gaattcgatggtaaccaaggcggggttttcgtatgctcattccagtggtcatctgatctagccaag

SEQ ID NO: 3 - DNA, 66 bp, Artificial Sequence
  Synthetic
gaattcgatggtaaccaagggggtgtttgtttttgtagtcattgttggtcatctgatctagccaag

SEQ ID NO: 4 - DNA, 64 bp, Artificial Sequence
  Synthetic
gaattcgatggtaaccaacccaatgtgtttcgtttgttttcatttgtcatctgatctagccaag

SEQ ID NO: 5 - DNA, 66 bp, Artificial Sequence
  Synthetic
gaattcgatggtaaccaatttccgtattgttggtttgtgtttgtgtcgtcatctgatctagccaag

SEQ ID NO: 6 - DNA, 64 bp, Artificial Sequence
  Synthetic
gaattcgatggtaaccaacccggacatagcgctgtgtgtgaattcgatggtaaccaacatccga

SEQ ID NO: 7 - DNA, 66 bp, Artificial Sequence
  Synthetic
gaattcgatggtaaccaagtgtattgtttattcttctgtttcttattgtcatctgatctagccaag

SEQ ID NO: 8 - DNA, 66 bp, Artificial Sequence
  Synthetic
gaattcgatggtaaccaaggaatatttggggttttttgcttctggacgtcatctgatctagccaag

SEQ ID NO: 9 - DNA, 66 bp, Artificial Sequence
  Synthetic
gaattcgatggtaaccaagcaatttgtatttttgtgtatttattttgttcatctgatctagccaag

SEQ ID NO: 10 - DNA, 66 bp, Artificial Sequence
  Synthetic
gaattcgatggtaaccaatgcgcttatgttttttttattttttatgtttcatctgatctagccaag

SEQ ID NO: 11 - DNA, 66 bp, Artificial Sequence
  Synthetic
gaattcgatggtaaccaatgatgttatttttttgtgcttttattcttttcatctgatctagccaag

SEQ ID NO: 12 - DNA, 67 bp, Artificial Sequence
  Synthetic
gaattcgatggtaaccaaggtcacttttttatgtcgcttaaatgttggtcatctgatctagccaagc

SEQ ID NO: 13 - DNA, 20 bp, Artificial Sequence
  Synthetic
ttnngtnttttnntnnttnt

SEQ ID NO: 14 - DNA, 18 bp, Artificial Sequence
  Synthetic
cttaagctaccattggtt

SEQ ID NO: 15 - DNA, 19 bp, Artificial Sequence
  Synthetic
gcttggctagatcagatga